SEQUENCE LISTING (21 sequences)

SEQ ID NO: 1 - protein, 6 aa, synthetic construct
HHHHHH

SEQ ID NO: 2 - protein, 14 aa, synthetic construct
MHHHHHHENLYFQS

SEQ ID NO: 3 - protein, 438 aa, Thermobifida halotolerans
MSAGRFHLGWFLEGSNAQAWGQPWTGSSGANWARQDLFVDMARALERACFDYILLEDSSYVADSYGGSMEIYLKNAIATPRQDPVVVASLLVQATSRIGIVPTLGTFQYHPYQLARLIGTLDQVSDGRIGWNMVTGSSDRAAQNFGMPGMPEHDLRYDMADEFMEVVFGLWDSWEPDAILADRGSGVFADPAKVRPIDFRGEHYSSRGPLNSGPLPQGRAVIAQAGGSPRGRRFASRYAETIIASVPGVAAMKAYRDDVRSLMEEHGRKPDDCKVLFLVSPVIGQTREEAAELKRQRDLAALERAEISLAHLSKVTNIDFSVFDIDQPLGDVELSTNGHQSTLADFLKAGRDRTLREAIATRSGNESVPLVGTPDDVAAQMGEVMEEVGGDGFLFVSPNLNWRTVTEVADGLVPALQRRGLTRSAYSHAHLRDNLLEF

SEQ ID NO: 4 - protein, 399 aa, Thermoanaerobacter ethanolicus
MWETKINPNKVFELRCKNTTYFGIGSIKKIKDILEVLKNKGINNVILVTGKGSYKASGAWDVVKPALETLGFKYSLYDKVGPNPTVDMIDEAAKIGRETGAKAVIGIGGGSPIDTAKSVAVLLEYTDKNARELYEQKFIPEKAAPIIAINLTHGTGTEVDRFAVATIPEKNYKPAIAYDCLYPMYAIDDPSLMTKLDKKQTIAVTIDALNHVTEAATTLVASPYSVLMAKETVRLIVRYLPAAVNDPENLVARYYLLYASALAGISFDNGLLHLTHALEHPLSAVKPEIAHGLGLGAILPAVVKAIYPSVAEVLAEVYSPIVPGLKGLPAEAEYVAKKVEEWLFKVGCTQKLSDFGFTKEDIPTLVRLAKTTPSLDGLLSNAPVEATEAVIAKIYEESF

SEQ ID NO: 5 - protein, 507 aa, Thermobifida halotolerans
MTVYAAPGTTGSIVEYRSRYDNFIGGEYVPPVKGDYFANVTPVTGRPFTEVARSTAEDVDRAVDAAEKAAPAWGRTSPAERALVLNRIADRVEASLEKLAVAETWENGKPVRETLAADLPLVVDHFRYFAGAIRAQEGRTSQIDDTTVAYHFNEPLGVVGQIIPWNFPLLMAAWKLAPALAAGNAVVLKPAEQTPASIHLLVELVADLLPAGVLNVVNGFGAEAGRPLAQHSRVRKVAFTGETTTGRLIMQYASENLIPVTLELGGKSPNIFFSDVAAERDAFYDKALEGFALFALNSGEVCTCPSRALVQADIYDAFLADAVERTRAIRTGNPLDTETMIGAQASNDQLEKILSYIDIGRQEGAKVLLGGERLEHEGDLAGGYYVAPTVFEGHNGMRIFQEEIFGPVVSVTRFDDYADAVAIANDTLYGLGAGVWSRSGTTAYRAGRDIQAGRVWVNNYHAYPAHAAFGGYKQSGIGRENHHMMLQHYQQTKNLLVSYSDNALGLF

SEQ ID NO: 6 - protein, 515 aa, Thermobifida halotolerans
MLNLSVLLEDGARNRPERDAIVFGDMRLNYAVVDMIANQVANLLVSRGVRPGDRVAVACPNVPYFPFVYFGALKAGAVVVPLNVLLTPREIEYHLRDSGAKALFAFTGTPELPLGERAWKAFHEVAECEVYIDLPATAGATESAIEGAETFWAALNGQPGEFESVRTEADDTAVIIYTSGTTGRPKGAQLTHNNLLLNAVVSSDLFDHSPDDHDVFLTVLPLFHIFGQTTMMNAALYRNATMVLMPRFDGDEALSLMEQEGVTVFAGVPTMYWGLLNTRNEHDIPAIAAKLHTAVSGGASLPSEVARQVKERFGIGILEGYGLSETSPVVSFNNPKRKAKPGSIGLPVWGVEMKLVDENFTTVGGEGPGEIAVRGHCVMKGYHNRPEVNAQVIRDGWFRTGDIARRDEEGFYFIIDRSKDMIIRGGYNVYPREIEEVLMTHPQVSLAAVVGVPHDRHGEEVKAFVIRAPEATVTEEELIAWSRERLAAYKYPRLVEFRDELPMTATGKILKRELR

SEQ ID NO: 7 - protein, 508 aa, Thermobifida halotolerans
MQLSLATILAEAARRHPDRTAIITDDAHVSYATLWEDSLRYAGALRSLGVVPGDRVALLAGNVVDFVRCYYGILAVGGTVVTVPPMLSAEEAAFLLSDTGARLVLAQDMFAALARAAVGDGDVQVRTTGEPHRPDALESVAAAAEPLPSHVSRSPDDVAVIFYTSGTTGTPKGALLTHLNLVMNATVNAFDVHPMSGDDVVMGSLPLFHTFGQSVALNTTFRVGATLVLQERFDPDEAIRMMREHGATIFDGVPTMFVRLAEAAANHSELPDLRFCVSGGASLPEVVLRRFEEAFSTTIYEGYGLSETSPTASTNQPLYGARTGSVGHPIWGVDVEIARADIDERVELLPRGEQGEIVVRGHNVFAGYLNRPEATAEAVVDGWFRTGDIGFRDDEGFIHIVDRKKDLIIRGGFNVYPREVEEVLLRFPGISQVAVVGLPDAELGEEVCAVVVPEKKGAVDPAELQSWARERLGRHKYPRRVEIVTELPLGPSFKVLKRELRARFGGKS

SEQ ID NO: 8 - protein, 598 aa, Thermobifida halotolerans
MREYTSPAKGDLPPDTRLTNTVFARAAEEPEAVMFRRQENGQWRDVTCARFRDDVVDVAKALIAAGIEHGDRVALMSRTRYEWTVIDYAVWTVGGVTVPIYDSSSEEQVHWILSDSDSKAVFVETAEHAALVKAVAGDLPELGDVWQIEGGGLGELIGSGSGVDDSAVEERRTAVTLDDLATLIYTSGTTGRPKGCELTQRNLTFDVMSVNSGPMHQVFSLPDRSSLLFLPLAHSLARIIQVGCVETKTVMGHFPSTGPELIDALATFKPTFLLAVPRVFEKVYNKAEQKAVSEGKGKIFHAAAETAIAYSKALDSGKVGFGLRAKHALFNRLVYGKILAAVGGEAKYAVSGGSALGDRLGHFFRGIGLTILEGYGLTETSAPATANDPATNRIGTVGRPIPGTSIRIAEDGEVLVKGDNVMRGYWKNPEATKNAFTEDGWYRSGDIGELDDDGFLRITGRKKEIIVTAGGKNVAPAVIEDRIRGHAIVSQCMVVGDNRKFVAALVTIDPEAFEFWKKQHGKTGTVAELVDDPDLRAAVQEAVDDGNRAVSKAESVRKFAILPVDFTEAGGQMTASLKLKRHVIAKQFSAEIDALYAD

SEQ ID NO: 9 - protein, 721 aa, Thermobifida halotolerans
MSTGFSAEAEDFGRTVADVLADLWGDATAAPDGDLARLWKTAAAQGWFELASERALDFLVAAVRELGRRACPLPVVDAYVAGLLLGADHAERVASGRLRPVVAVVDDPGTDTADHCEAAPHATHVLLVHPGSGRVRLAPISRVEPTPGLAVPAWSRVGVAEPETELRAEAALIDDALAVWTLGLAVRALGAAGRGHELAVAHAATREQFGRVIGGFGAVQQRVAACHIEVGAAEALVKDAVGHGLAESGEWTLAAALAVRYVGDTATSVMFGAQHTLGAIGFFEEHEAAWLFRRVHADVARLRAAARHATGVSRALVEQGRSLPSFHLGEAAEEFRSRIRALLAEHRVVDGHGRQDHDRERLRAAMAERGLFSLGWPVEEGGREAGVAEQAVLNEEMKYARGPVDRAMSAAMLLGHSILKHGTPEQKAEFLPLIRRGEMRFCLGYSEPEAGSDLASLRTGAVRDGDHWVVNGQKSWTTRAHTASHVWLAVRTDPDAVPRHAGITIFLVPMDTPGITVQRHTALSGEVSCTVFYDDVRVPDSARVGEVNGGWRVITDALAAERVVMGGVAATLLRQLDDLLEHLRTDPERMLGADGSGKRERLESLAARLQAARALVLEATRAMAGGGGSDLAAPMAGVLGGELAEDFGQGVLELLGPDAALGADVPRAPGGGVFERALRTSPMFVIGGGTNDIQRAIVARGLGLPREERAARGRGAGCGPR

SEQ ID NO: 10 - protein, 270 aa, Thermobifida halotolerans
MAQMTTPTAEELAASGLGLAVDGAVARITLSRPRRRNAMTGRTWTMLARIGQTLPEAVRIVVVGGAGPTFSSGIDLDMFRPGDIDGEPTPLSLLADHPDDTAPLERAIAEYQEGFLWLRRADIVSVAAVRGHAIGAGFQLALSCDIRVLSDTARLCMKEPALGLVPDLTGTKPLVELVGVNRAVELCLTARTIDAEEAARLRLAEQVVADSELEPAVDELVAQLLAVPADAARATKELLLQAGRNDLETQARAERAAQLARLVAMAGAAR

SEQ ID NO: 11 - protein, 289 aa, Thermobifida halotolerans
MGELRFDGRVAVITGAGHGLGRSHALGFAARGARVVVNDLEEPAAAAVAERIRAHGGSALTHTGDISTEEGAQSLVDAANDTFGRVDVIVNNAGILRDRSFKNISTREWDEVLAVHLRGAFLVSRAAFEHLREHGYGRIVNTTSPSGLFGNFGQSNYSAAKMGVVGLTKTLAIEGAKYDINVNAVAPIAYTRMTEDLFPADAAAVLSPEKVTPLVLWLAHEDCPTTGEVYSVGGGRIARVFVAEGPGVVLEHTTAEEIQDNWPDVNAERPYVIPRNLGEQTEIHIRGLL

SEQ ID NO: 12 - protein, 717 aa, Nocardia seriolae
MTENMIGWEKDADGIVVLTMDDPNQGANTMNQLYKDSMRATVDRLVAEKDDITGVVITSAKKTFFAGGDLKNMMQTTKADAAAIMEELTFIKSDLRRLETLGKPVVSAINGAALGGGLEITLATHHRIAADVKGLQLGLPEVSLGLLPAGGGVTRITRMLGIANGLMGVLLQGNKFNAQKAKATGLIHEVVSSVEELVPAAKAWIKANPEGGVQPWDVKGYKIPGGTPSSPALAANLPAFPANLRKQLKGQNMPAPQAIMAAAVEGAQVDFDNASLIESRYFVSLLTGPVAKNMIQAFFFDRSHINNGGSRPKDVPKREIKKVGVIGAGMMGAGIAYVTAKAGIDVVLKDVTIEAANHGKDYSVKLEEKALSRGKTTEEKSKTLLDRITPTADAADFAGVDFVIEAVFENPELKAKVFGEIEDIVDADALLGSNTSTLPITLLANGVKRAEDFIGIHFFSPVDKMPLVEIIKGEKTSDEALARVYDYTLAIRKTPIVVNDSRGFYTSRVIGKFINEAIMMLGEGVDPQTIEQAGLQAGYPAAPLKLSDELNFTTMQKIYKETAEAIVAAGGEIDAASGATAQILDKLVGEFDRKGKAGGAGFYDYVDGKATGLWAELRSTFKTSTALPEGVTLQDLKDRMLFAEAIETQKCFDENVLTSTADANIGSIFGIGFPAWTGGTHQFIVGYPGGQEAFVARADELAAKFGSRFEVPASLRK

SEQ ID NO: 13 - protein, 247 aa, Tepidiphilus sp.
MTTRIALVTGGMGGLGEAICIKLAQLGYRVVTTHSLNNKNAPQWLADMKAKGFDFAAYPADVADFDSCKACVEKIVQEIGPVDVLVNNAGITRDMTLRKMTKADWDAVLRTNLDSVFNMTKQVIDGMVERKWGRVINISSVNGQKGAFGQTNYAAAKAGMHGFTKSLALEVARYGVTVNTVSPGYIGTKMVLAIPQEILDSKILPQIPMGRLGKPEEIAGLVAYIASDEAAYMTGANIAINGGQHMY

SEQ ID NO: 14 - protein, 322 aa, unidentified
MFLLALVTSLIGTGMFTTPVHAAGQFIRDTAPDGRVYKLYIPSGYNGSTPLPLVVMLHGCTQNPDDFAAGTEMNVYAEQNNFLVAYPEQPSSANLNKCWNWFDSNHQSRGRGEPASIAGVVEDVKRNYSVDSRRVYAAGLSAGGAMSVIMGATYPDVFAAIGVGSGLEYKAATSMTSAYMAMINGGPDPVQQGNLAYQAMGSHARVVPVIVFHGTSDYTVYPVNGHQVISQWAQTNDRAGDGVDNNHIDDQADVTMNGSVPNGRTYTRYLYKDQNGNVVMEKIMVNGMGHAWSGGSTAGTYTDPAGPEASSMMWSFFVNHPK

SEQ ID NO: 15 - protein, 563 aa, Pseudomonas thermotolerans
MPTRHQPAVNTLPAPAAFMNAQNAVVGLRGRDLLSSARDIALHGLRHPLHGTRHLLSFGKQLGRVLLGDTLYAPNPGDVRFADPTWQLNPLYRRGLQAYLAWQKQLVQWIDESDLSADDRTRALFLASLLSDALAPSNSLLNPLAIKELFDTGGGSLLRGLRHLIDDLLHNDGLPSQVNKQAFEVGKNLATTPGSVVFRNEMLELIQYRPMSEKQYERPLLIVPPQINKFYIFDLSPEKSFVQYALKNDLQVFMISWRNPDARHREWGLSSYVQALDEALEVCRAITGSKAVNLLGACAGGLTAAALMGHLQARRQLRKIASASYAVSLLDSQIESSPALLFVDEQTLEAAKRRSYQAGVLDGRDLAKVFAWMRPNDLIWNYWVNNYLLGRQPPAFDILYWNNDNTRLPAALHGDLLDIFKHNPLTRPGGLEVCGTPIDLQRVTVDSFCVAGINDHITPWDAVYRSLLLLGGERRFILSNAGHIQAILNPPGNPKAHYFENGRLSADPRAWYYDARKVEGSWWPEWLAWIQARSGEQRPTRMSIGNARYPALEAAPGTYVLVR

SEQ ID NO: 16 - protein, 316 aa, Thermus thermophilus
MFSLAGCGQLGSGPKERKSLVPRTVVMGSFTHPEGTRNFRLYLPPRTAELPLMVLLHGCTQTAQDFALGTSMDRLADQLGFAVLYPEQLPEANGSRCWNWFDPSHQDRGSGEPAILADMAAGLAQKYGLYPSQMAVAGLSAGGAMAVILAATYPDRFAFLGVVAGLEYKAAKSLPEAFAAMATGGPDPSQQGLAVCQSWGRKAYRLPLLVFHGDSDTTVNPVNGEQVYQHWLVAHQACGSAQDLREQRFTVNEGGRSATVRQMEDGQGILAEFWLVQGMGHRWPGGNALGSYTDPQGPEASRRIAERWTAILKGGE

SEQ ID NO: 17 - protein, 550 aa, Halomonas titanicae
MPSVTPKILPAAASATNPALMIGDLLEAGVRMAGDNQIVYRDQSRHSYERFRERVHQLAHTLTSQGVQAGDVVAVLDWDSHRYLECFFAIPMIGAVLHTVNVRLAPEQILYTMDHAEDVFVIVHEEFVPLLEPLADKLPQVRGYLLCQETPQTVDTTLPLVGEFESLLSQQPTHYDFPTFDENAVATLFYTTGTTGNPKGVFFTHRQLVLHTLNESSAFQAPGFELLNRDKVYMPITPMFHVHAWGVPYTATLMGATQVYPGRYEPEMLVKLLVSEKVDFSHCVPTLLNMVVSAEAITSKQVDLAGWKVLVGGSALTESLASKAWALGIDTRSAYGMSETCPLLTADILPQDIGDADFETQLPWRCKAGLPIPLVKLQVVDADGEPLPNDGKSVGEVRVQAPWLTQAYYKEEKRSEELWRNGWLHTGDVGSLDERGFLTISDRIKDVIKTGGEWLSSLELESYISQCPGIAEVAVIGVTDDKWGERPAALAVPADLNNPPTTEAVQAFLEQFVAQGKLNRWGIPSMIRFVDEIPKTSVGKLDKKRIRTEI

SEQ ID NO: 18 - protein, 556 aa, Halomonas titanicae
MSEHANAAVLRGPALEGLDQYDSVTDVFHNAVTRFKDKPAFSCMGKTLSFADLDRLSADFAAWLQHETDLVPGDRIAIQLPNVLQFPVAVFGALRAGLVVVNTNPLYTEREMANQFKDSNAKAIVILANMADKLEKVLDKTDIKHVLVTQLADLHDVPKRWLINAVVKHVKKMVPAYSLPSAVGFRDALKKGASLSHTEVKRSHDDLAALQYTGGTTGMPKGAMLTHRNLIANMLQAREAIGTHLTDGEELVIAPLPVYHIYTFTVNCLFLMETGNHSLLITNPRDLDGFIKELKGLPFSAFIGLNTLFNALCNRDDFKQLDFSKLKLTISGGMALTKAAAQRWEKTTGCPIAEGYGLTETSPIVSFNPTDSIQLGTIGKPVAGTAVKVVDADGNDMALGEPGELCVQGPQVMKGYWQREDETRDAIDEDGWFRTGDIAVLQDDGYIRIVDRKKDMILVSGFNVYPNEVEDVVAAHPDVLESAAVGVPDENAGEAIKLFVVSKNDQLDEKTLRDWCKKELTGYKVPKFIEFRDELPKTNVGKVLRRQLRDEEPSKT

SEQ ID NO: 19 - protein, 512 aa, Halomonas titanicae
MSHNLFETFAAKMRERAEADFITTRDGRRYRYADALTISAQLAGALTELGVKQGDRVAVQVDKSPEAILLYLACLRIGGVYLPLNTGYTGDEIRYFLNDAEPALFVCRPKIEEQALSLAAETGCPAVVTLGSAADGSLMETAQAAVPREDIVALGERDLAAILYTSGTTGRSKGAMLTHKNLASNAKTLAKAWHFSADDRLIHALPIFHTHGLFVACNVTLMAGASMLFLPKFDADVIFEELPRGTVMMGVPTFYTRLVQDERLTPEATANMRLFVSGSAPLTAETHEAFEAKTGHAILERYGMTETNMNLSNPYDGARRAGTVGMPLPGVEMRITNRETGAEVPFGEIGMLQIRGPNVFIGYWRMPEKTREELLDDGFFITGDLAMVDEQGYVHIVGRDKDLVISGGYNVYPKEVEQVIDELDQVAESAVIGLPHPDFGEGVTAVVVRQQGADLGENQLEEEKVITHLDGRLAKYKQPKRVFFVDELPRNTMGKVQKNELRKRFNDTYQTP

SEQ ID NO: 20 - protein, 505 aa, Halomonas titanicae
MVNLSAFIAFHARQRPEHTAIIYGEERVSYKAFDQRIRRLAALLKQRGIGKDDIVAVFMKNSPAFLEVAFATSHLGAVFLPVNFRLAANELAHILNDAGAKLLFIDEELTSVCEIDQHVVVVDAMAQHDSRRLSSPDWPLPDAVPRRSDQLFRLMYTSGTTARPKGVMHSYENFYWKCMDHVIALNLTAEDKLLTVGPLYHVGAFDLPGVAVLWRGGTICLHREFNAEDALASIERHQLSCAWTAPVMLNQILSVESPERFDLSSLRWCIGGGEKTPESRIRDFTRVFTSARYIDGYGLTESCSGDTLMAPGMEIEKIGSTGRALAHVEICICDEQGKPLPSNTDGEICLRGPKVTRGYWNDPEKTAESFFDDWFRTGDIGHLDGDGFLFVTDRRKDMILTGAENVASSEVENVIYLLPQVAEAAVVGLPDEQWGERLVAVVVLREGKTLSYTQLEHHCRGHLANFKVPKQLLISEGLPRNPSGKILKRELREMYAQPSFDSSHP

SEQ ID NO: 21 - protein, 507 aa, Halomonas titanicae
MNVANYLAVNAVRFPHRIATYQGDVPALSYAELACQARRWATFFARQGLAPGERVALFLPNSPVYIELLCGAWYAGLVVVPVNAKLSVAEVGYIADDSGSCIVFCERDDVAQALTDTLVLPPMLPEGLVHEESFDRPVFRAPDAPAWIFYTSGTTGKPKGATLTHLNLAAMSACYAANIEPVTAQSRLVHFAPMSHGSGLYIVPYLMAGGANVIPGSLKFSGESFVSTLAGHDHVSMFLAPTMIRRVLDYLAAHQPEYSAAHIATITYGGGPMYAKDLEEALRVFGHRLVQIYGQGESPMTITFLGKGQHHQLTPQCLSGDAEGSPLLPVGQSHPQLEVTIQDQEGSCLPCGQEGEVCVRGDMVMAGYWNNPAATQETLRDGWLRTGDIGHLDPQGFLYLTDRLKDVIISGGTNIYPREVEEVLLQFPGVKEVSVVGQRDAEWGEVVMAFIACSVSLDTTALDQFCLNRMARFKRPKSYHFLTDLPKNAYGKILKTELRKRLDGVTS

(19) United States
(12) Patent Application Publication
(10) Pub. No.: US 2026/0098283 A1
(43) Pub. Date: Apr. 9, 2026

(54) In Vitro Bioproduction of Specific Chain Legnth Poly(hydroxyalkanoate) Monomers

(71) Applicant: Kimberly-Clark Worldwide, Inc., Neenah, WI (US)

(72) Inventor: Stephen Quirk, Alpharetta, GA (US)

(21) Appl. No.: 19/115,062

(22) PCT Filed: Sep. 28, 2023
(86) PCT No.: PCT/US2023/033962

§ 371 Date: Mar. 25, 2025

Related U.S. Application Data
(60) Provisional application No. 63/411,673, filed on Sep. 30, 2022.

(51) Int. Cl. C12P 7/6418; C12N 9/00; C12N 9/02; C12N 9/04; C12N 9/10; C12N 9/88; C12P 7/6427

(52) U.S. Cl. CPC C12P 7/6418; C12N 9/0006; C12N 9/0008; C12N 9/001; C12N 9/0077; C12N 9/1029; C12N 9/88; C12N 9/93; C12P 7/6427; C12Y 101/01135; C12Y 101/01136; C12Y 101/01211; C12Y 101/99002; C12Y 103/08008; C12Y 114/15003; C12Y 402/01017; C12Y 602/01003

(57) ABSTRACT

An enzymatic process and system are disclosed for producing homogeneous chain length poly hydroxyalkanoate monomers and/or polymers having a chain length of at least eight carbons from a long-chain fatty acid or precursor thereof. The process also results in specific homogenous chain length polyhydroxyalkanoate monomers and/or polymers. The long-chain fatty acid or precursor thereof can be contacted in vitro with an enzyme or a mixture of enzymes to produce the homogeneous chain length polyhydroxyalkanoate.

### CROSS REFERENCE TO RELATED APPLICATIONS

This application claims priority to the benefit of U.S. Provisional Application No. 63/411,673, filed Sep. 30, 2022 which is expressly incorporated herein by reference in its entirety.

### BACKGROUND

It has been estimated that over 300,000,000 metric tons of petroleum-based polymers are being produced each year with global production continuing to increase. A significant portion of the petroleum-based polymers are used to produce single-use products, such as plastic drinking bottles, straws, packaging, absorbent articles, including wearable absorbent articles, and other polymer waste. Most of these plastic products are discarded and do not enter the recycle stream. In addition, the rapid rise of nano-and microplastics found in organisms that are deleterious to those organisms has greatly increased, causing further concern. Regardless of the plastic, it is increasingly important to develop new polymers or methods to provide true circularity of use in order to mitigate health and environmental impact of petroleum based plastics.

Biodegradable polymers produced from renewable resources (also termed “biopolymers”) hold great promise for reducing the global accumulation of petroleum-based plastics in the environment. One such class of biopolymers are the polyhydroxyalkanoates (PHA). Much work has been accomplished on the small chain PHA family, most notably the polyhydroxybutyrate (PHB) polymers including poly-3-hydroxybutyrate (P3HB), poly-4-hydroxybutyrate (P4HB), polyhydroxyvalerate (PHV), polyhydroxyhexanoate (PHH), polyhydroxyoctanoate (PHO) and their copolymers. However, while PHAs exhibit thermoplastic properties that are in some ways similar to some petroleum-based polymers and may therefore represent potential replacements for petroleum-based polymers such as polypropylene and polyethylene, existing characterized PHAs do not yet inherently contain all properties, such as processability, melting temperature, crystallinity, and the link, to form true one-to-one replacements for petroleum based polymers. For instance, existing PHA synthesis is limited to C1-C8 PHAs, whether via synthesis or commercially, which has hindered research into PHA-polypropylene/polyethylene comparable polymers.

Polyhydroxyalkanoates are synthesized using a variety of bacterial and archaea genera, including Halobacillus, Bacillus, Salinobacter, Flavobacterium, Chromohalobacter, Halomonas, Marinobacter, Vibrio, Pseudomonas, Halococcus, Halorhabdus, Haladaptatus, Natrialba, Haloterrigena, and Halorussus, rendering PHAs viable replacements for petroleum based polymers as they are fully biorenewable and biocompatible. The polyhydroxyalkanoate serves as an energy sink for these organisms. Production of polyhydroxyalkanoate polymers by the above microorganisms involves a three-step enzymatic mechanism that begins with acetyl coenzyme A. In forming PHB, the first step is catalysis of acetyl-CoA by PhaA (a β-ketothiolase) to form β-ketoacyl-CoA. This in turn is converted in a NADP-dependent reaction into R-3-hydroxyacyl-CoA by the PhaB enzyme (a β-ketoacyl-CoA reductase). The final step, catalyzed by PhaC (a PHB synthase), is the polymerization of R-3-hydroxyacyl-CoA into PHB. Said another way, the final step of the pathway involves the polymerization of hydroxyalkanoic acid monomers into a polyhydroxyalkanoate polymer via a polyhydroxyalkanoate polymerase. However, existing methods are only capable of producing five straight chain PHAs:

hydroxypropionate, hydroxybutyrate, hydroxyvalerate, hydroxyhexanoate, and hydroxyheptanoate, and require purification if a single chain length is needed. In addition, existing processes often fail to provide straight chain and/or homogenous PHA polymers.

Thus, a need exists for systems and processes that can produce a more extensive range of chain length biopolymers in vitro. It would be a further benefit if the biopolymer is suitable for use in consumer products and industrial processes. Additionally or alternatively, it would be economically and environmentally advantageous to produce homogeneous, straight chain, and/or single polymeric species, preferably without further purification or separation. It would be an additional benefit to provide an in vitro enzymatic process for forming such bioplastic polymers.

### SUMMARY

In general, the present disclosure is directed to an enzymatic process for producing homogeneous chain length polyhydroxyalkanoate monomers and/or polymers having a chain length of at least eight carbons. The process includes contacting a long-chain fatty acid or precursor thereof, in vitro with an enzyme or a mixture of enzymes, and producing homogeneous chain length polyhydroxyalkanoate monomers and/or polymers having a chain length of at least eight carbons.

In one aspect, the homogeneous chain length polyhydroxyalkanoate monomers and/or polymers have a carbon chain length that is equal to a central carbon chain length of the long-chain fatty acid or precursor thereof. Additionally or alternatively, the central carbon chain length of the long chain fatty acid is any one of the following carbon chain lengths: C6, C7, C8, C9, C10, C11, C12, C13, C14, C15, C17, C18, C19, C20, C21, C22, C23, C24, C25, C26, C27, C28, C29, and C30, and/or the central carbon chain length of the long-chain fatty acid precursor is configured to produce a long chain fatty acid having any one of the following central carbon chain lengths: C6, C7, C8, C9, C10, C11, C12, C13, C14, C15, C17, C18, C19, C20, C21, C22, C23, C24, C25, C26, C27, C28, C29, and C30. Moreover, in an aspect, the homogeneous chain length polyhydroxyalkanoate monomers and/or polymers is any one or more of the following carbon chain lengths: C8, C9, C10, C11, C12, C13, C14, C15, C17, C18, C19, C20, C21, C22, C23, C24, C25, C26, C27, C28, C29, and C30.

In another aspect, the process is carried out in one vessel, or alternatively, the process is carried out in more than one vessel.

Additionally or alternatively, in an aspect, the long-chain fatty acid precursor is a long-chain alkane, a long-chain alcohol, or a long-chain aldehyde. Moreover, in one aspect, the enzyme or mixture of enzymes contains at least one enzyme selected based upon the central chain length of the long-chain fatty acid. In yet a further aspect, the process includes a second long-chain fatty acid or precursor thereof having a different chain length from the long-chain fatty acid or precursor thereof, where the produced homogenous chain length polyhydroxyalkanoate is a copolymer having segments of the long-chain fatty acid or precursor thereof and second long-chain fatty acid or precursor thereof.

Further, in another aspect, the process includes one or more further steps of contacting the long chain fatty acid or precursor thereof in vitro with the enzyme or the mixture of enzymes. In one aspect, the process includes contacting the long chain fatty acid or precursor thereof in vitro with a first enzyme followed by one or more subsequent enzymes in a stepwise manner. In yet another aspect, the process includes contacting the long chain fatty acid or precursor thereof in vitro with two or more enzymes simultaneously. In one aspect, the enzyme or the mixtures of enzymes is purified from an extremophilic microorganism, where, in an aspect the microorganism is a bacteria of genera: Halomonas, Lihuaxuella, Lysobacter, Alteromonas, Arthrobacter, Azospirillum, Empedobacter, Desulfovibrio, Halobacillus, Halobacteriovorax, Haloechinothrix, Halomarina, Halorussus, Haloterrigena, Isoptericola, Marinobacter, Methyloligella, Micromonospora, Natronococcus, Nocardiopsis, Paracoccus, Roseivivax, Saccharomonospora, Shewanella, Alicyclobacillus, Natranaerobius, Halobacteriaceae, Hyphomonas, Amycolatopsis, Georgenia, Acidothermus, Thermobifida, or a combination thereof. Additionally or alternatively, the microorganism is an engineered microorganism that has been genetically modified to secrete a specific enzyme and/or is at least one type of a naturally occurring microorganism that naturally encodes a specific enzyme.

In an aspect, the homogeneous chain length polyhydroxyalkanoate monomers and/or polymers have a linear carbon chain. In yet a further aspect, the homogeneous chain length polyhydroxy alkanoate monomers and/or polymers is at least about 90% homogeneous.

Additionally or alternatively, in an aspect, the enzyme or the mixtures of enzymes is a thermophilic enzyme. In an aspect, the thermophilic enzyme is temperature tolerant from about 40° C. to about 120° C. In yet another aspect, the process is performed at a temperature range from about 40° C. to about 80° C. Further, in one aspect, the process utilizes enzymes that co-function effectively in the same environment characterized by the same or similar pH and temperature. Moreover, in an aspect, the process is an organism-free process, where the long-chain fatty alcohol or precursor thereof is contacted with the enzyme or mixture of enzymes in an environment substantially absent any bacteria that secrete the same enzyme or the mixture of the same enzymes.

The present disclosure is also generally directed to an uncharacterized polyhydroxyalkanoate that is a polyhydroxyalkanoate having a carbon chain length greater than C8, where the polyhydroxyalkanoate is a linear chain polymer substantially absent any side chain pendant polymers.

The present disclosure is also generally directed to a process of producing homogeneous chain length polyhydroxyalkanoate monomers and/or polymers having a chain length of at least eight carbons. The method includes contacting a long-chain fatty acid with a long chain fatty acid CoA ligase/synthetase to obtain a long-chain fatty acid acyl-CoA, contacting the long-chain fatty acid acyl-CoA with a long chain acyl-CoA dehydrogenase, followed by a long-chain-enoyl-CoA hydratase, followed by a hydroxyacyl-CoA dehydrogenase or a long-chain-3-hydroxyacyl-CoA dehydrogenase to obtain a long-chain acetoacetyl-CoA, and contacting a long-chain acetoacetyl-CoA with an acetoacetyl-CoA reductase to obtain a hydroxyacyl-CoA for polymerization into a polyhydroxyalkanoate.

In one aspect, the long chain fatty acid CoA ligase/synthetase is purified from Thermobifida halotolerans. Additionally or alternatively, in an aspect, the alcohol dehydrogenase is a fungal long-chain alcohol dehydrogenase purified from the group of fungal genera comprising: Aureobasidium, Macroventuria, Lophium, Tothia, Trichodelitschia, Westerdykella, Didymosphaeria, Viridothelium, Delitschia, Zopfia, Myriangium, Rhizodiscina, Saccharata, Aaosphaeria, Amniculicola, Byssothecium, Aspergillus, Meira, Dissoconium, Lizonia, Aureobasidium, Morchella, Sodiomyces, Tilletiopsis, Jaminaea, Ceraceosorus, Testicularia, Tilletiopsis, Violaceomyces, Rhizopus, Alternaria, Hesseltinella, Neurospora, Ramularia, and Rhynchosporium.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is an illustration of potential starting molecule classes for the biosynthesis of specific PHAs;

FIG. 2 is an illustration of an enzyme reaction for the creation of long-chain fatty acid-CoA molecules that are used as substrate for the formation of specific chain length PHAs;

FIG. 3 is an illustration of enzyme reactions for the in vitro formation of homogeneous PHAs;

FIG. 4 is chart illustrating substrate chain length preference distribution for the three T. halotolerans long chain fatty acid-CoA synthetase enzymes;

FIGS. 5A(i) to FIG. 5D(v) illustrate sequences of the various enzymes used in the examples and figures;

FIG. 6 is a graphical representation of a reduction in the concentration of fatty acid in the initial reaction of Example 1 (Arachidic acid (C18), Open circles; Myristic acid (C12), closed circles);

FIG. 7 is a graphical representation of the formation of pyrophosphate in the initial reaction of Example 1 (substrates: Arachidic acid (C18), Open circles; Myristic acid (C12), closed circles); Excitation wavelength 316 nm, emission wavelength 456 nm;

FIG. 8 is a graphical representation of the formation of precipitating PHA of Example 1 as measured by the increase in optical density (due to scattering) at 650 nm (Arachidic acid (C18), Open circles; Myristic acid (C12), closed circles);

FIG. 9 is a graphical representation of the depolymerization of the formed PHA of Example 1 as measured by the decrease in optical density (due to scattering) at 650 nm (Arachidic acid (C18), Open circles; Myristic acid (C12), closed circles);

FIG. 10 is a graphical representation of the formation of pyrophosphate in the initial reaction containing equal mass amounts of Arachidic acid (C18) and Myristic acid (C12) of Example 2;

FIG. 11 is a graphical representation of the formation of precipitating PHA as measured by the increase in optical density (due to scattering) at 650 nm of Example 2 containing equal mass amounts of Arachidic acid (C18) and Myristic acid (C12);

FIG. 12 is a graphical representation of the depolymerization of the formed PHA as measured by the decrease in optical density (due to scattering) at 650 nm containing equal mass amounts of Arachidic acid (C18) and Myristic acid (C12) of Example 2;

FIG. 13 is a graphical representation of the formation of pyrophosphate in the initial reaction utilizing hexacosanoic acid (CH3(CH2)₂₄COOH) as the sole substrate of Example 3;

FIG. 14 is a graphical representation of the formation of precipitating PHA as measured by the increase in optical density (due to scattering) at 650 nm utilizing hexacosanoic acid (CH3(CH2)₂₄COOH) as the sole substrate of Example 3;

FIG. 15 is a graphical representation of the depolymerization of the formed PHA as measured by the decrease in optical density (due to scattering) at 650 nm utilizing hexacosanoic acid (CH3(CH2)₂₄COOH) as the sole substrate of Example 3 (PHA depolymerase from T. thermophilus, closed circles; L. thermophila, open circles);

FIG. 16 is a graphical representation of the formation of pyrophosphate in the initial reaction utilizing heptananoic acid (CH₃(CH₂)₅ COOH) as the sole substrate of Example 4;

FIG. 17 is a graphical representation of the formation of precipitating PHA as measured by the increase in optical density (due to scattering) at 650 nm of Example 4 (reactions initiated with heptananoic acid (CH₃ (CH₂)₅COOH) as the sole substrate);

FIG. 18 is a graphical representation of the depolymerization of the formed PHA as measured by the decrease in optical density (due to scattering) at 650 nm of Example 4 (reactions initiated with heptananoic acid (CH₃(CH₂)₅COOH) as the sole substrate. PHA depolymerase from L. thermophila);

FIG. 19 is a consensus maximum likelihood tree showing the evolutionary relationship between the five most unique fatty acid-CoA ligases expressed by Halomonas titanicae;

FIG. 20 is a graphical representation of a reduction in the concentration of fatty acid in the initial reaction of Example 5 (Octanoic acid (C6), closed circles; Myristic acid (C12), closed squares; Behenic acid (C20), open circles);

FIG. 21 is a graphical representation of the formation of pyrophosphate in the initial reaction of Example 5 (substrates: Octanoic acid (C6), closed circles; Myristic acid (C12), closed squares; Behenic acid (C20), open circles); Excitation wavelength 316 nm, emission wavelength 456 nm;

FIG. 22 is a graphical representation of the formation of precipitating poly (hydroxyalkanoate) of Example 5 as measured by the increase in optical density (due to scattering) at 650 nm (Octanoic acid (C6), closed circles; Myristic acid (C12), closed squares; Behenic acid (C20), open circles); and

FIG. 23 is a graphical representation of the depolymerization of the formed poly (hydroxyalkanoate) of Example 5 as measured by the decrease in optical density (due to scattering) at 650 nm (Octanoic acid (C6), closed circles; Myristic acid (C12), closed squares; Behenic acid (C20), open circles).

Repeat use of reference characters in the present specification and drawings is intended to represent the same or analogous features or elements of the present invention.

### DETAILED DESCRIPTION

### Definitions

The terms “about,” “approximately,” or “generally,”, when used herein to modify a value, indicates that the value can be raised or lowered by 10%, such as 7.5%, such as 5%, such as 4%, such as 3%, such as 2%, or such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain aspects, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

As used herein, the term “biodegradable” or “biodegradable polymer” generally refers to a material that degrades from the action of naturally occurring microorganisms, such as bacteria, fungi, archaea, and algae; environmental heat; moisture; or other environmental factors. The biodegradability of a material may be determined using ASTM Test Method 5338.92.

As used herein, the term “enzyme” generally refers to an enzyme that includes but is not limited to the following: native enzyme, purified enzyme, wildtype enzyme, modified enzyme, or combination thereof.

As used herein, the term “microorganism” includes bacteria, fungi, archaea, and algae, wildtype or modified, that expresses or produces one or more enzymes discussed herein

As used herein, the terms “polyhydroxyalkanoate” or “hydroxyalkanoate” generally refer to a chemical family of biopolymers that includes but is not limited to the following members: the polyhydroxybutyrate (PHB) polymers including poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), as well as uncharacterized PHAs having carbon chains of greater than C8, as will be discussed in greater detail below, and each of their monomers and copolymers.

### DETAILED DESCRIPTION

It is to be understood by one of ordinary skill in the art that the present discussion is a description of exemplary aspects only and is not intended as limiting the broader aspects of the present disclosure.

In general, the present disclosure is directed to an enzymatic process for producing homogeneous poly (hydroxyalkanoates), PHAs, in a manner that results in a single molecular species of the polymer. Namely, the present disclosure has surprisingly found that the unique in vitro synthetic route discussed herein allows for the production of a single polymeric species, removing the need to purify the produced PHA polymer pool to length homogeneity. In addition, the in vitro method discussed herein is also surprisingly capable of producing novel PHAs that have not been described or that cannot be produced by other methods, such as PHAs having a chain length of greater than eight carbons. Namely, without wishing to be bound by theory, the present disclosure has surprisingly found that by carefully selecting a combination of enzymes and process conditions, novel PHA polymers of homogeneous but varied chain lengths (e.g. having greater carbon chain lengths than those currently believed possible) can be produced without requiring a multi-step or multi-container process, and is even able to avoid in-vivo processes that limit the production of varied PHAs.

Namely, the present disclosure has surprisingly found that specific chain length, including long chain, starting materials can be enzymatically converted to acetyl-CoA precursors, which can then be subsequently enzymatically converted to corresponding specific chain length/long chain length PHAs. For instance, an in-vitro process according to the present disclosure utilizes one long chain substrate species to be enzymatically converted in-vitro, ensuring that the resulting PHA polymer is completely homogeneous. In addition, utilizing the process as discussed herein, PHA long chain copolymers can be created by starting with two or more different chain length substrates. In such an aspect, the final PHA copolymer will reflect the composition and ratio of the input substrate molecules. Thus, the method of the present disclosure provides an alternative to existing bioprocesses that result in the formation of mixed PHA size classes as well as the ability to produce longer length PHA polymers instead of short to medium chain polymer that are available via current in vivo bioreactor processes.

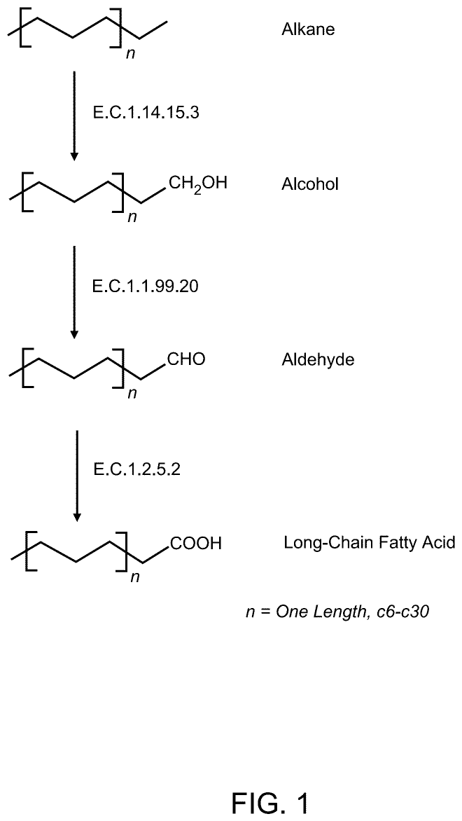

In one aspect, the starting material of the process as discussed herein can be any long chain fatty acid, or a single chain length precursor thereof. For instance, the long chain fatty acid can contain a C6-C30 carbon chain, excluding the carbons of the carboxy terminus. (such a carbon chain length can also be referred to by the long chain fatty acid n-2 carbons to account for the cleaved CH₂COOH which will be discussed in greater detail below). As illustrated in FIG. 1, in one aspect, the long-chain fatty acid itself may be a suitable precursor.

However, depending upon the precursor sources available, the aldehyde, alcohol, or alkane precursor to a long-chain fatty acid may also be utilized. Referring to FIG. 1, and as may be understood in the art, and as a specific example only, conversion from a long chain alkane to a long chain alcohol can be performed by an alkane monooxygenase (such as E.C. 1.14.15.3 for example only); the formation of a long chain aldehyde from a long chain alcohol can be catalyzed by an Alkan-1-ol dehydrogenase/oxioreductase (such as E.C. 1.1.99.20 for example only) or by a long-chain alcohol dehydrogenase (such as E.C. 1.1.1.2 for example only); and the formation of the long chain fatty acid from the aldehyde can be catalyzed by an aldehyde dehydrogenase (such as E.C. 1.2.5.2 for example only). As illustrated, when starting with a long chain fatty acid no enzymatic conversion would be needed to enter the PHA synthesis cycle. Nonetheless, as noted, even when starting with an alkane precursor (such as, an alkane obtained from a post-consumer and/or recycled source, in one aspect only), the process as discussed herein can be a fully enzymatic process. Furthermore, as will be discussed in greater detail below, even if starting with an alkane, alcohol, or aldehyde, it is possible to carefully select the chain length, as well as perform a “one-pot” reaction by careful selection of enzymes that are able to co-exist without negative interactions or side-reactions.

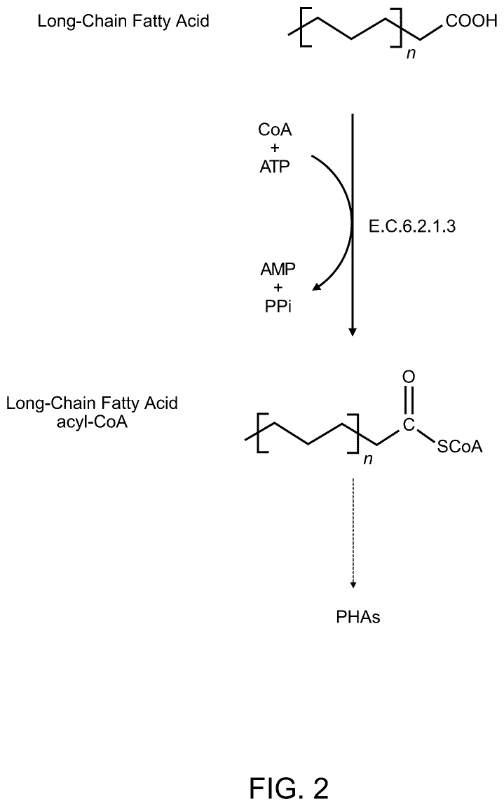

Nonetheless, as illustrated in FIG. 2, after the long chain fatty acid with a desired chain length n is obtained or produced, it is converted into the Coenzyme A moiety. In one aspect, the conversion into the coenzyme A moiety is conducted by a long chain fatty acid-CoA synthetase (may also be referred to in the art as ligases or synthases, such as E.C. 6.2.1.3 for example only). This reaction and the specific cofactor requirements for the reaction are shown in FIG. 2. Moreover, as known in the art, in nature the formation of PHB begins with formation of acetoacetyl-CoA. However, as illustrated in FIG. 2, the process of the present disclosure allows the formation of PHAn beginning instead with the formation of a long chain fatty acid acyl-CoA where n is the chain length of the fatty acid. Thus, allowing long chain and homogeneous PHA's to be accurately and efficiently formed.

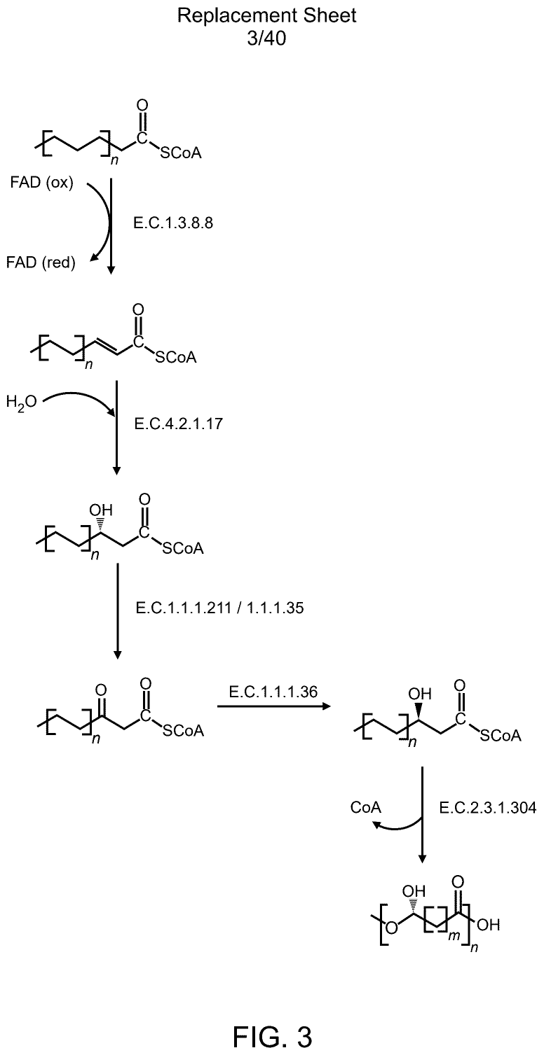

Subsequently, there is a four-step process illustrated in greater detail in FIG. 3 to convert the long chain fatty acid-CoA into the form that can be polymerized by a PHA polymerase (which may also be referred to as a synthetase, synthase, lligase). The first reaction is that of a long-chain acyl-CoA dehydrogenase (such as E.C. 1.3.8.8 for example only) which allows the production of a long-chain trans-2,3-dehydroacyl-CoA. This in turn is the substrate for the second reaction using a long-chain-enoyl-CoA hydratase (such as E.C. 4.2.1.17, for example only) which forms the long chain hydroxyacyl-CoA. The third reaction forms a long chain oxoacyl-CoA (using a hydroxyacyl-CoA dehydrogenase, such as E.C. 1.1.1.35 for example only) or alternatively, a specific long-chain-3-hydroxyacyl-CoA dehydrogenase (such as E.C. 1.1.1.211, for example only). Finally, the long chain hydroxyacyl-CoA is formed by acetoacetyl-CoA reductase (such as E.C. 1.1.1.36 for example only).

Once the long chain hydroxyacyl-CoA is formed, the long chain hydroxyacyl-CoA is polymerized via a PHA polymerase (such as 2.3.1.34, for example only) to form the specific, homogeneous chain length n PHA monomer. Moreover, as illustrated in FIG. 3, the formed PHAn will be in multiples of n, the chain length of the monomer long-chain-3-hydroxyacyl-CoA. For example, if the entire reaction was run with a C12 fatty acid, the resulting PHAn will have length, n, in multiples of 12 (e.g. n=24, 36, 48, etc. for each polymerization event). Of course, as discussed above, in a further aspect, the polymer can be a copolymer of two specific inputs. For instance, if n=C12 and b=C18 fatty acids (or precursors thereof) inputs are utilized, the resulting PHA polymer will be a copolymer of the C12 and C18 monomer species.

While it should be understood that, in one aspect, the enzymatic processes may be performed utilizing enzymes from a single source, it should also be clear that the process as discussed herein provides a further benefit over in-vivo processes, namely, that enzymes purified from different sources may be combined based upon the inputs and kinetics, and/or multiple forms of a single enzyme can be utilized. For instance, by utilizing the process of the present disclosure, an optimal enzyme for each step in the process can be used, even if the original sources is from widely different bacteria. Namely, the process as discussed herein is highly capable of producing specific PHAs, without additional metabolic side reactions taking place, as are generally present in in-vivo approaches, nor is there metabolic energy spent on growth and cell division, increasing efficiency. Moreover, by utilizing the enzymatic process of the present disclosure, any or all the enzymes can be engineered to increase properties that make the enzyme more compatible with the overall process, such as site directed mutations to alter stability, kinetic rate, the like, or combinations thereof.

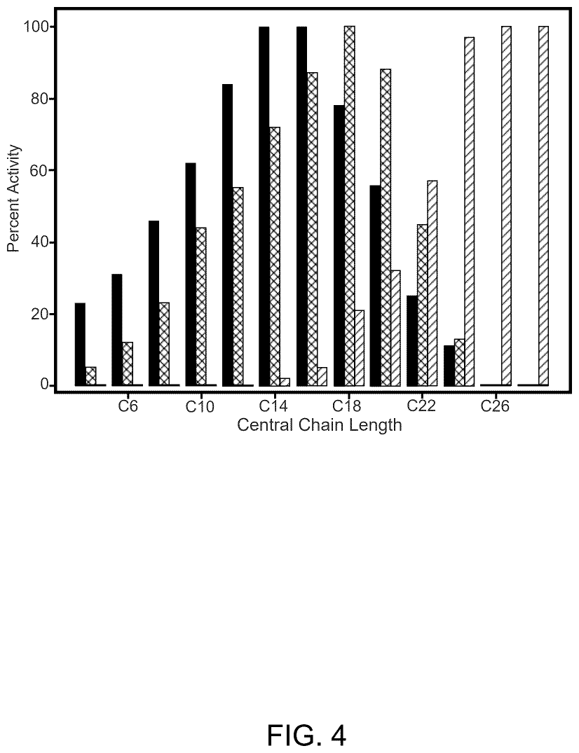

For instance, in one aspect, two or more enzymes purified from a single species can exhibit preferences for disparate chain length reactions, particularly the long chain fatty acid-CoA synthetase enzymes discussed above in regards to FIG. 3. Namely, referring to FIG. 4, three T. halotolerans long chain fatty acid-CoA synthetase enzymes were purified. As illustrated, each of the enzymes has a preference for different central chain lengths (where the carbon chain length refers to the central portion of the molecule as noted above, e.g. C14 refers to CH₃(CH₂)₁₄COOH. WP_068688876: cross-marked bars, WP_068692787: black bars, WP_06869753.1: hatched bars). Thus, in one aspect, the process as discussed herein contains enzymes from two or more organisms in order to improve stability, kinetic rate, or other performance factors, based upon the carbon chain length of the precursor.

Moreover, as will be discussed in greater detail below, in one aspect, thermophilic enzymes can be utilized as reactions at higher temperature can, in one exemplary aspect only, increase thermodynamic driving force. But as noted above, any source of enzyme(s) can be employed if they catalyze the reaction for the length of the fatty acid carbon chain.

Nonetheless, in one aspect, the present disclosure is generally directed to contacting one or more homogeneous long-chain fatty acid monomers, having a chain length (excluding the carboxy terminus) of C8 or greater, or an alkane, alcohol, or aldehyde precursor thereof, in vitro with an enzyme or a mixture of enzymes, and producing one or more C8 or greater PHA bioplastic monomers or polymers. In one aspect, the long-chain fatty acid monomers, having a chain length (excluding the carboxy terminus) of C8 or greater, or an alkane, alcohol, or aldehyde precursor thereof can be obtained from post-consumer materials, such as post-consumer personal care products, food industry products, packaging, post-consumer medical products, post-consumer industrial products, and other articles. The post-use product can also include components of post-industrial use and/or other polymer waste.

In an aspect, the post-use products may contain contaminants. The process and systems herein can eliminate or reduce such contaminants. Post-use products can have contaminants that include, without limitation, mesophilic pathogens, such as, without limitation, viruses, bacteria, fungi, and protozoans, can be rendered non-pathogenic by disclosed methods. As utilized herein, the terms “mesophile” and “mesophilic” refer to organisms that naturally exist in environmental conditions at which humans generally co-exist with the organism, including near human body temperature (e.g., from about 20° C. to about) 45°, a saline content in water of from about 5 to about 18 parts per thousand (also referred to as mesohaline), about one atmosphere pressure (e.g., from about 20 kPa to about 110 kPa), and near neutral pH (e.g. from about pH 5 to about pH 8.5, also referred to as neutrophiles or neutrophilic). Typical bacterial pathogens encompassed herein can include those commonly found in human stool such as, and without limitation to, those of a genus Streptococcus, Bifidobacterum, Lactobacillus, Staphylococcus, Clostridium, Enterobacteriaceae, or Bacteroides.

However, it should be understood that, in one aspect, the precursors may be obtained from a non-recycled or non-post-consumer product, or may be produced specifically for the method as described herein. For instance, in one aspect, one or more of the precursors can be formed from a method directed to formation of the precursor.

Nonetheless, regardless of the source of the precursor, the present disclosure is generally directed to combining an enzyme, or a mixture of enzymes, particularly selected for carrying out one or more reactions suitable for converting a long-chain fatty acid monomer to a homogeneous long-chain PHA monomer, with the long-chain fatty acid monomer(s), having a chain length (excluding the carboxy terminus) of C8 or greater, or an alkane, alcohol, or aldehyde precursor thereof. In addition, it is believed that this mechanism can avoid the generation of microplastics and nanoplastics which is a further benefit over non-biobased polymers, as nano and microplastics are a growing concern as they may be more toxic than intact petroleum-based polymers.

Furthermore, as noted above, in one aspect, the reactions of FIG. 1 (as necessary if not starting with a long-chain fatty acid) to 3 can be run together or separately, according to the design of the overall process or as dictated by the careful selection of enzymes. Namely, as noted above, and without wishing to be bound by theory, the present disclosure has found that, by carefully selecting enzymes that require (or thrive) in similar conditions, and which do not utilize any intermediates produced during the reactions of FIGS. 1 to 3, the method discussed herein, starting with the long-chain fatty acid or precursor thereof, can be conducted as a “one-pot” reaction, allowing further improvements in efficiency, speed, and footprint.

Nonetheless, in one aspect of the process disclosed herein, the long-chain fatty acid monomer(s), having a chain length (excluding the carboxy terminus) of C8 or greater, or an alkane, alcohol, or aldehyde precursor thereof, have any one or more of the following carbon chain lengths: C6, C7, C8, C9, C10, C11, C12, C13, C14, C15, C17, C18, C19, C20, C21, C22, C23, C24, C25, C26, C27, C28, C29, and C30. (where chain length, as discussed above, and when starting with a fatty acid precursor, is a chain length sufficient to produce a central chain length in the long-chain fatty acid monomer, which excludes the carboxy terminus, of the same length as the precursor). Exemplary fatty acids include arachidic acid (C18 central chain), myristic acid (C12 central chain), hexacosanoic acid (C24 central chain), according to the above listing. However, it should be clear that the fatty acid can be a fatty acid having a C6 to C30 central chain as discussed above.

In another aspect, the method of the present disclosure can be performed at a temperature range from about 40° C. to about 80° C. such as from about 45° C. to about 75° C., about 50° C. to about 70° C., or about 55° C. to about 65° C. For example, it can be performed at a temperature of about 40° C., about 45° C., about 50° C., about 60° C., about 65° C., about 70° C., about 75° C., and/or about 80° C. In one aspect, the method of the present disclosure can be carried out together in one vessel or in more than one vessel.

In yet another aspect, and as noted above, it was surprisingly found that the process can be fine-tuned by way of careful identification and selection of enzymes to suit a particular reaction in an industrial and/or laboratory scale process. For example, this may include careful calibration of reaction conditions such as, for instance, enzyme catalytic efficiency, pH optimum, or substrate discrimination. It can also include careful calibration of the overall reaction environment such as, for instance, selection of elevated temperature, high salt conditions, increased reaction pressure, and/or extremes of pH or cold. For example, thermophilic enzyme(s) may be selected if the reaction conditions are such that that temperature is elevated. Yet further, it can also be possible to conduct certain reactions of the present disclosure using multiple extreme conditions if polyextremophilic enzymes are utilized. In one aspect, thermophilic or thermotolerant enzymes can be utilized to produce PHAs from the long-chain fatty acid(s). In particular, thermophilic enzymes can be well suited due to the favorable thermodynamics of catalysis at elevated temperatures, however, any source of enzyme that catalyzes the reactions in, for example, FIGS. 1-3 can be utilized based on careful selection in consideration of the subsequent reactions and various reaction products.

Thus, in an aspect, the process can include one step of contacting the long-chain fatty acid(s) or precursors thereof in vitro with an enzyme or a mixture of enzymes. Namely, without wishing to be bound by theory, the present disclosure has found that, by carefully selecting enzymes that require (or thrive) in similar conditions, and which do not utilize any intermediates produced during the reactions of FIGS. 1 to 3, the method discussed herein, starting with the long-chain fatty acid(s) or precursor thereof can be conducted as a “one-pot” reaction, allowing further improvements in efficiency, speed, and footprint. However, in an aspect, the process can include one or more further steps of contacting the long-chain fatty acid(s) or precursor thereof in vitro with the enzyme or the mixture of enzymes by repeating the introduction of one or more enzymes for each of the one or more further steps discussed above and as illustrated in FIGS. 1 to 3. For example, the one or more further steps can be repeated more than two times, more than three times, more than four times, more than five times, more than six times, more than seven times, more than eight times, more than nine times, or more than ten times. For example, the one or more further steps can be repeated less than three times, less than four times, less than five times, less than six times, less than seven times, less than eight times, less than nine times, or less than ten times.

Furthermore, in another aspect, the process can include contacting long-chain fatty acid(s) or precursor thereof in vitro with a first enzyme followed by one or more subsequent enzymes in a stepwise manner. For example, the long-chain fatty acid(s) or precursor thereof in vitro can be contacted with a first enzyme then can be contacted by a second enzyme then by an optional one or more subsequent enzymes. In yet another aspect, the process can include contacting the long-chain fatty acid(s) or precursor thereof in vitro with two or more enzymes simultaneously.

In one aspect, the first enzyme, the second enzyme and/or one or more subsequent enzymes can be different from each other. In another aspect, the first enzyme, the second enzyme and/or one or more subsequent enzymes can include some enzymes that are different from each other while some enzymes may be the same or similar. For example, the one or more subsequent enzymes can include at least one additional enzyme that is different than the first enzyme.

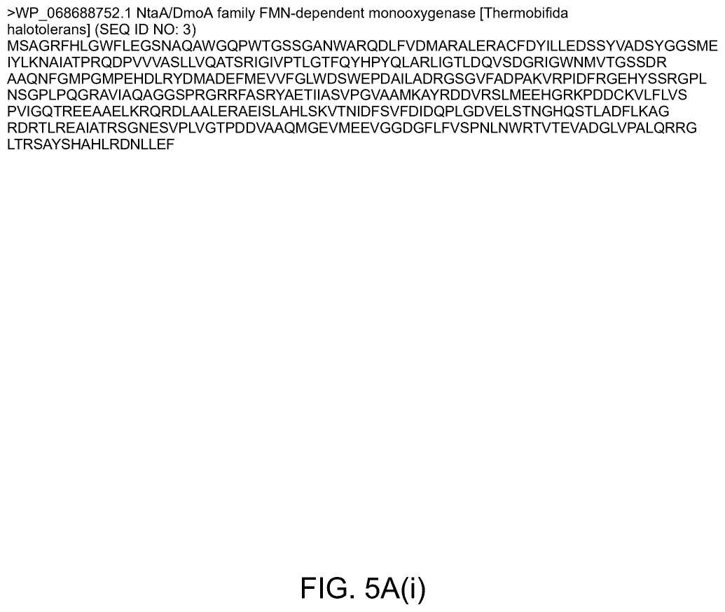

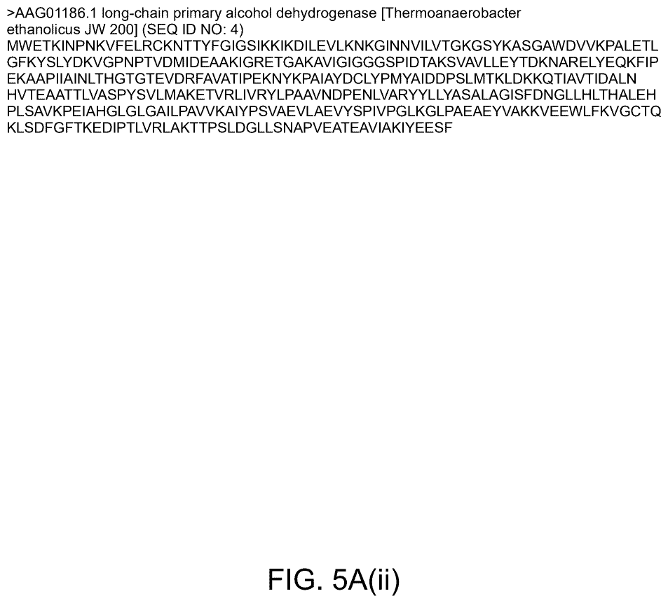

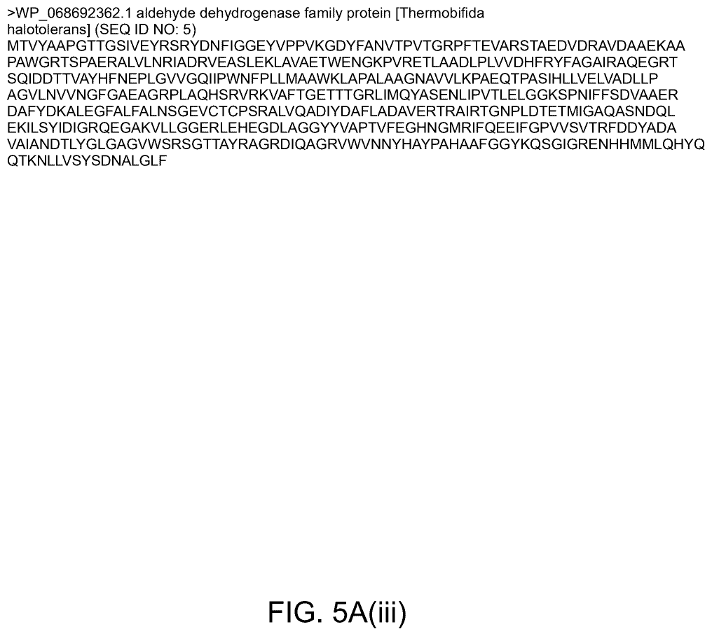

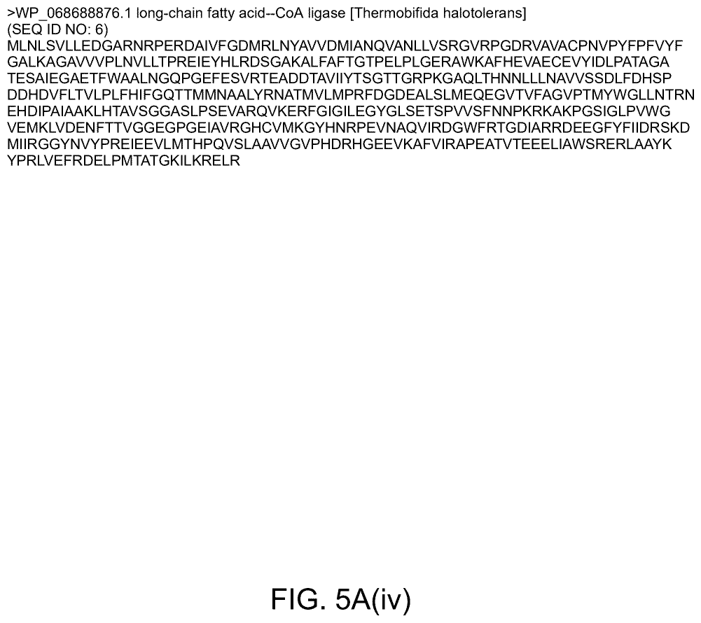

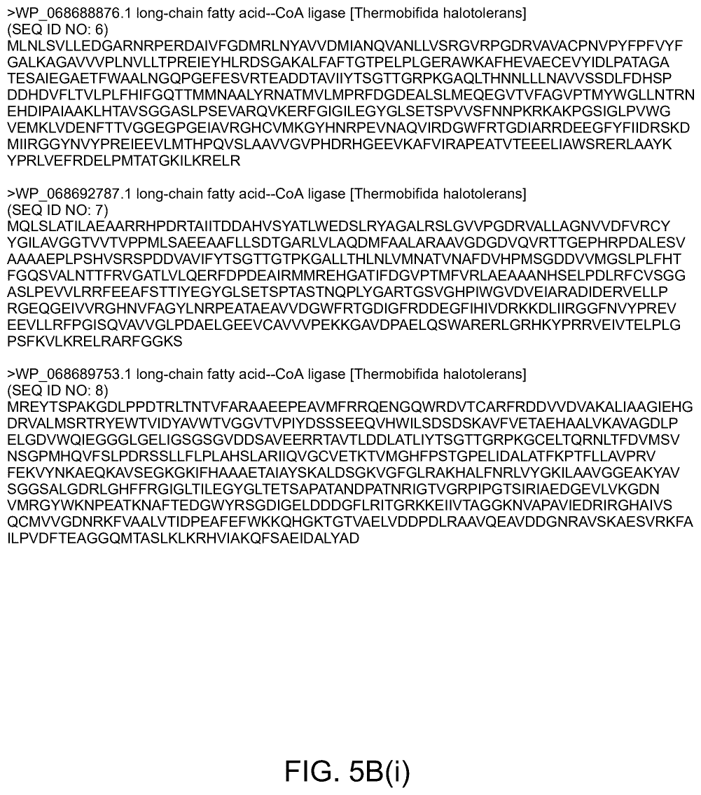

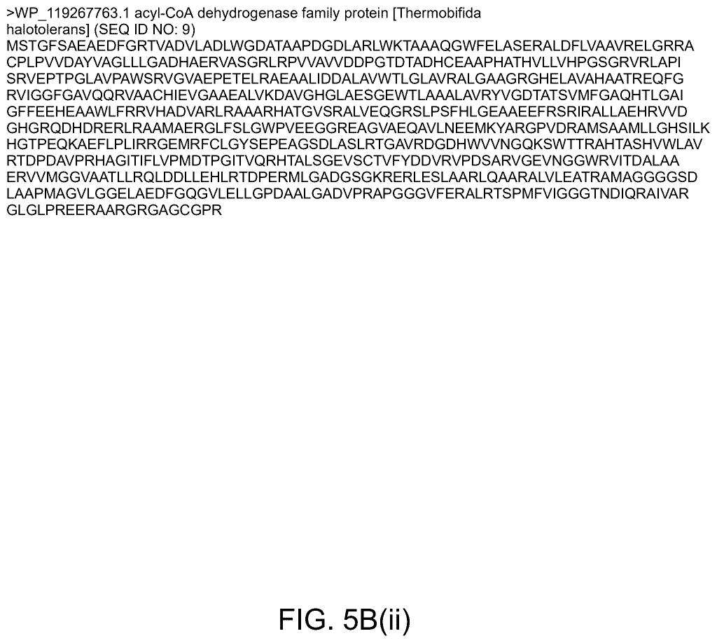

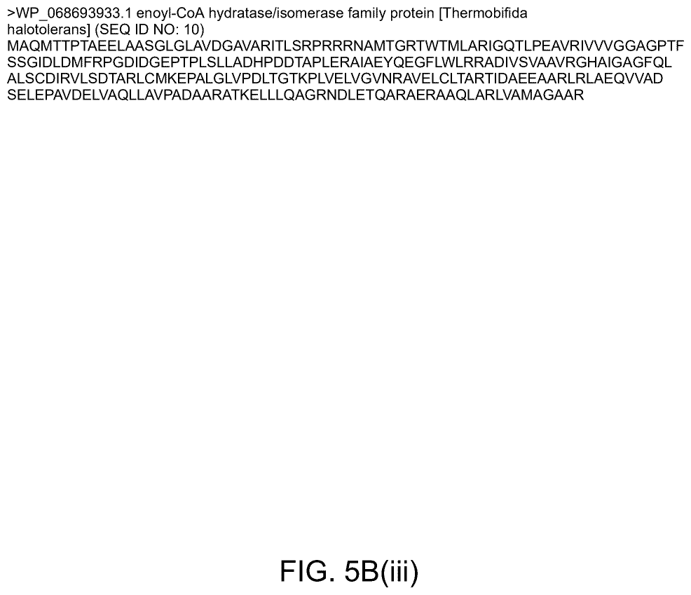

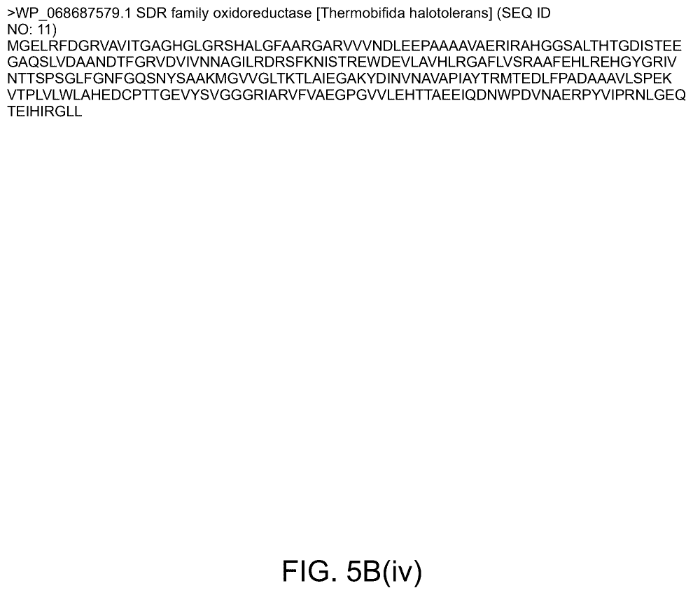

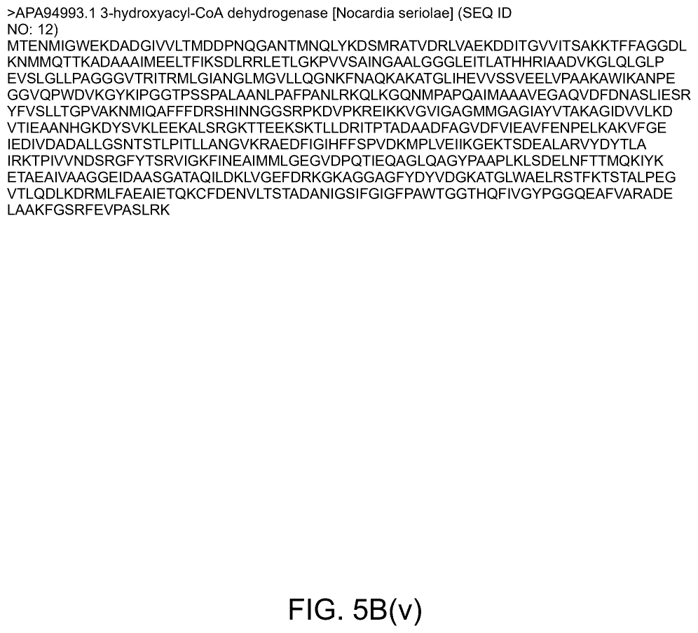

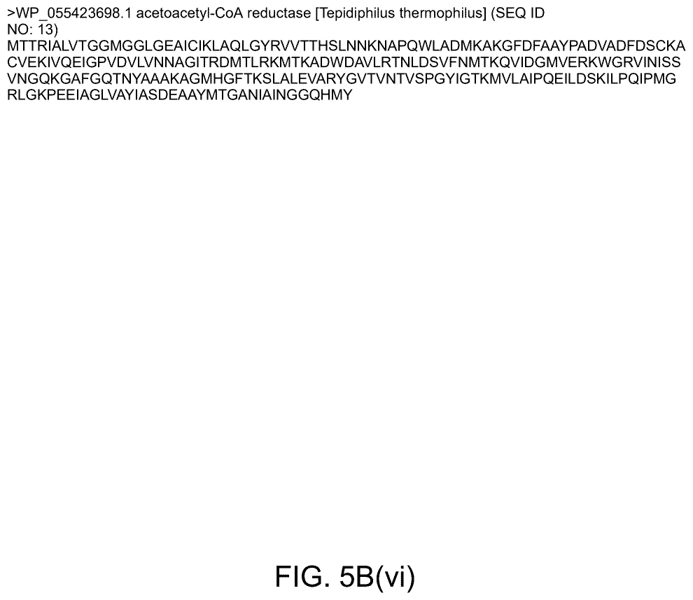

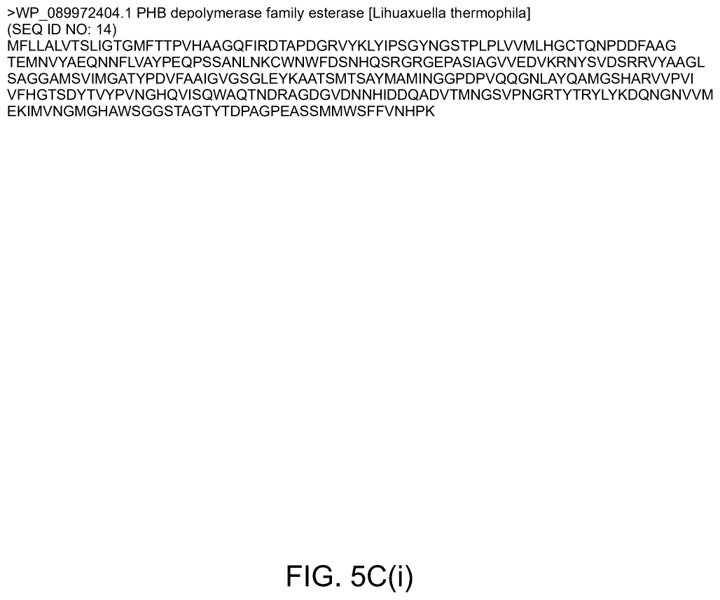

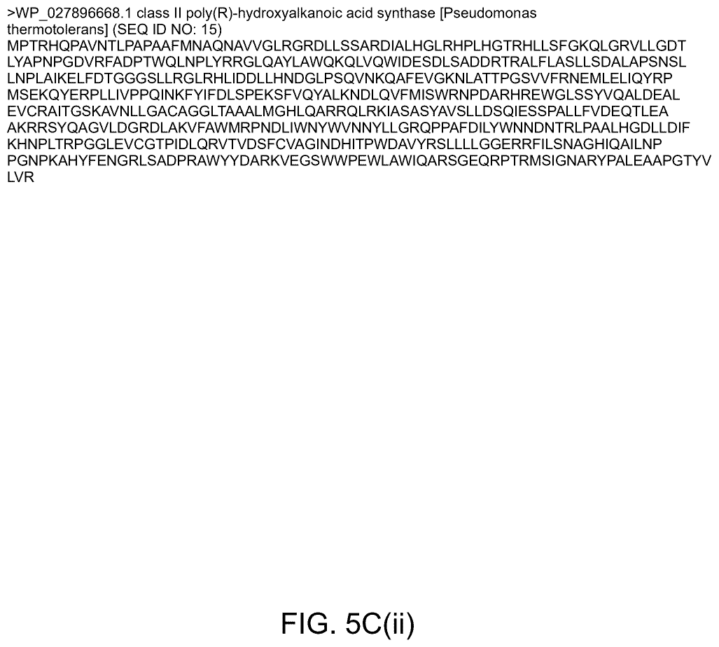

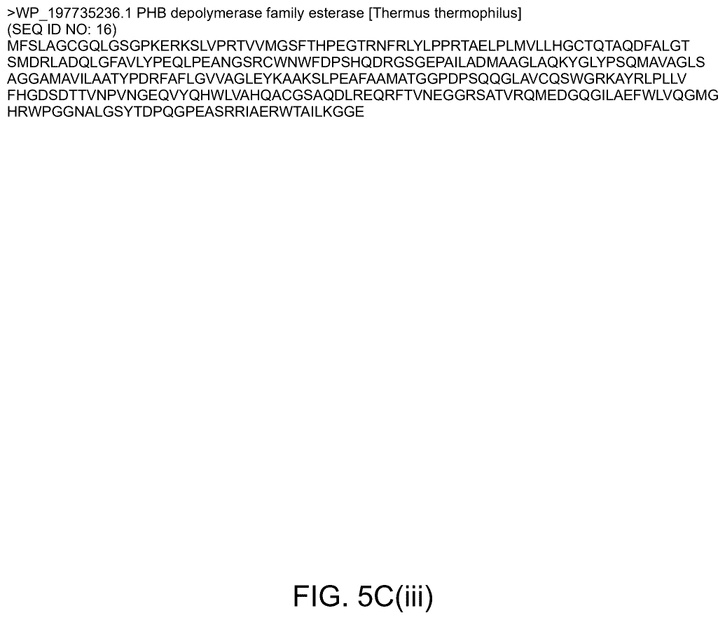

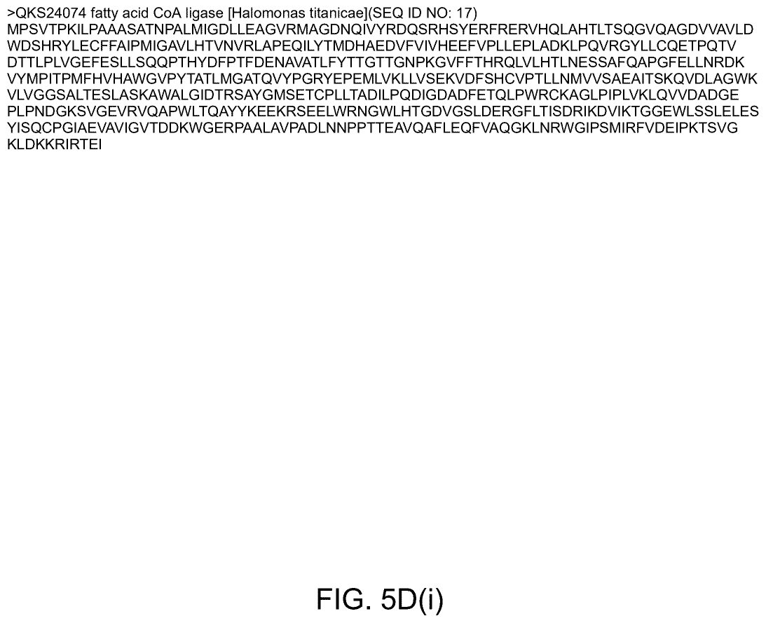

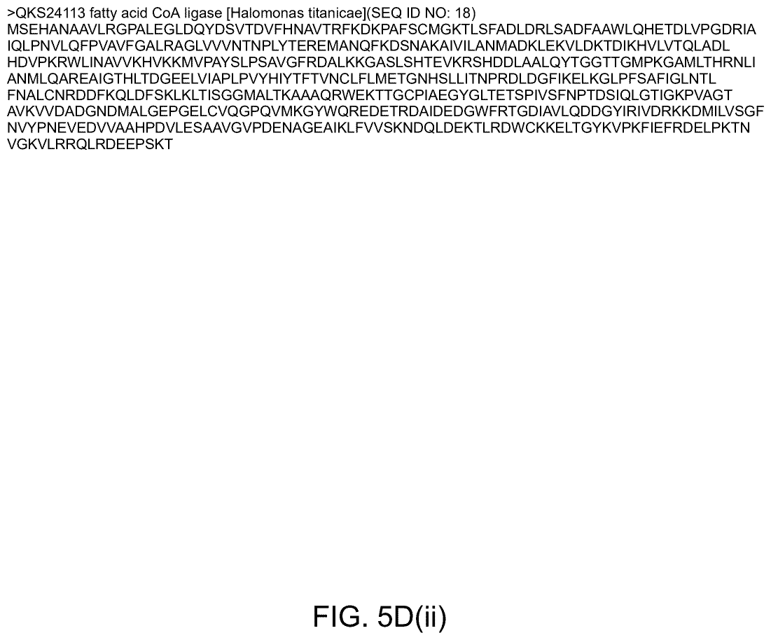

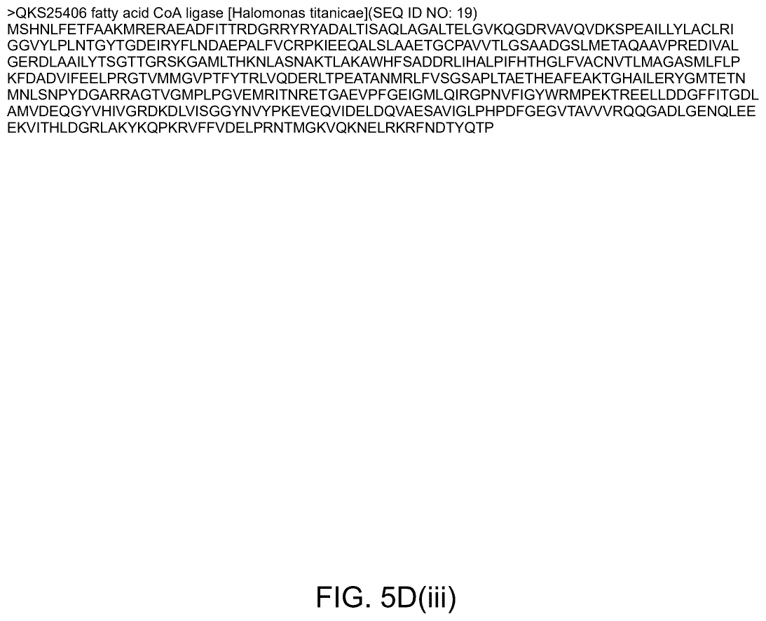

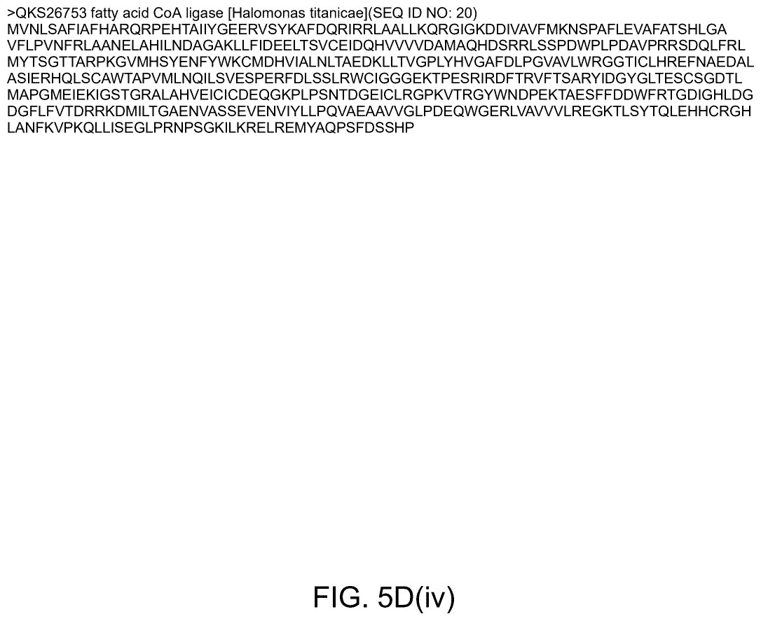

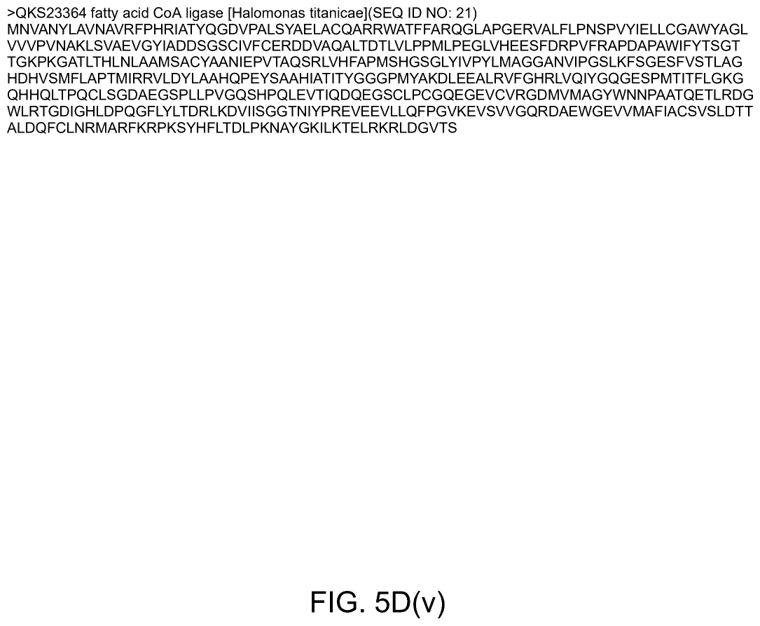

For example, sequences of enzymes that can be utilized in the process of the present disclosure are shown in FIG. 5. Albeit, only exemplary enzymes are shown, the present disclosure has discovered that by way of careful calibration of reactions and environmental conditions along with a thoughtful selection of the enzymes that catalyze a particular reaction, the process of the present disclosure can produce specific length, homogeneous PHA monomers and polymers. For example, engineered enzyme variants can also be suitable for a particular reaction or an overall reaction process.

In one aspect, the enzyme or the mixtures of enzymes can be purified from an extremophilic microorganism. For example, the microorganism can be a bacteria of the genera: Halomonas, Lihuaxuella, Lysobacter, Alteromonas, Arthrobacter, Azospirillum, Empedobacter, Desulfovibrio, Halobacillus, Halobacteriovorax, Haloechinothrix, Halomarina, Halorussus, Haloterrigena, Isoptericola, Marinobacter, Methyloligella, Micromonospora, Natronococcus, Nocardiopsis, Paracoccus, Roseivivax, Saccharomonospora, Shewanella, Alicyclobacillus, Natranaerobius, Halobacteriaceae, Hyphomonas, Amycolatopsis, Georgenia, Acidothermus, Thermobifida, or a combination thereof.

In one aspect, the enzyme or the mixtures of enzymes can include a thermophilic enzyme. For example, the thermophilic enzyme can be temperature tolerant from about 40° C. to about 120° C., such from about 50° C. to about 110° C., about 60° C. to about 100° C., or about 70° C. to about 90° C. Accordingly, in some aspects, an extremophilic enzyme for use in disclosed process and systems can be a thermophilic enzyme that exhibits a Topt of about 40° C. or greater, about 50° C. or greater, about 60° C. or greater, about 70° C. or greater, about 80° C. or greater, or about 90° C. or greater in some aspects. Exemplary thermophiles (and thermophilic enzymes produced thereby) encompassed herein can include, without limitation, Alicyclobacillus pomorum (WP-084453829), Amycolatopsis thermoflava (WP-123687648), Amycolatopsis thermalba (WP-094002797), Amycolatopsis rumanii (WP-116109633), Azospirillum thermophilum (WP-109324320), Deinococcus actinosclerus (WP-082689076), Fervidobacterium gondwanense (SHN54810), Gandjariella thermophila (WP-137812779), Georgenia satyanarayanai (WP-146237554), Hyphomanas sp. (HAO37884), Lihuaxuella thermophila (WP-089972404), Microbulbifer thermotolerans (P-197462976), Minwuia thermotolerans (WP-206420073), Rhodopseudomonas thermotolerans (WP-114356866), Rhodopseudomonas pentothenatexigens, (WP-114356866), Streptomyces thermovulgaris (WP-067396676), Thermanaeromonas toyohensis (WP-084666479), Thermoactinomyces sp. CICC 10523 (WP-198056464), Thermoactinomyces daqus (WP-033100012), Thermoactinospora sp. (NUT44302), Thermoactinospora rubra (WP-084965756), Thermobifida halotolerans (WP-068692693), Thermobifida fusca (WP-011290529), Thermobispora bispora (WP-206206594), Thermocatellispora tengchongensis, (WP-185055796), Thermochromatium tepidum (WP-153975900), Thermocrispum municipal (WP-028851041), Thermoflavimicrobium dichotomicum (WP-093229000), Thermogemmatispora carboxidivorans (WP-081839208), Thermogemmatispora aurantia (WP-151728970), Thermogemmatispora tikiterensis (WP-11243376), Thermogemmatispora onikobensis

(WP-084659191), Thermoleophilaceae bacterium (MBA2429278), Thermomonospora echinospora (WP-160147065), Thermomonospora cellulosilytica (WP-182704610), Thermomonospora amylolytica (WP-198679325), Thermostaphylospora chromogena (WP-093263254), Thermus thermophilus (WP-197735236), Thermus aquaticus (WP-053768217), Thermus islandicus (HEO42284). For example, at least one enzyme in step c) can be purified from Lihuaxuella thermophila.

In another aspect, the microorganisms from which the enzymes can be purified can be selected based on factors that include but are not limited to the following: easy and fast to grow in high density, do not require special media, aerobic, kinetically fast, stable, tolerant to high salt environment, tolerant in a temperature environment, able to produce readily purifiable enzymes, lack an unusual isoelectric point, do not require heightened biosafety measures, do not include Cysteine residues in excess, overall non-esoteric, available for purchase commercially, or a combination thereof.

However, while the enzymes have been discussed so far as being present in a solution, it should be understood that, in one aspect, the process may be performed utilizing one or more microorganisms that naturally express the discussed enzymes, or that have been modified to express the desired enzymes. For example, the microorganism can be at least one type of a naturally occurring microorganism that naturally encodes a specific enzyme for use the method discussed herein.

In addition to microorganisms that naturally express a certain enzyme gene, one or more genetically modified bacteria may also be selected that express an exogenous enzyme capable of performing specific reactions of the present invention. Yet further, the microorganism can be an engineered microorganism that has been genetically modified to secrete a specific enzyme for use in the method herein.

For example, any genus of bacterium or archaean can be matched with any enzyme that is expressed from a constitutive vector coupled with the correct signal sequence. In this aspect, any suitable gram positive or gram negative bacterium can be used to produce and secrete the enzyme of interest. In this manner, the enzyme can be customized based on environmental variables, the type and amount of post-use product, or combinations thereof. In addition, the sequence of the enzyme can be matched to the environment by selecting one of the known sequences (e.g. NCBI database) or with a fully or partially engineered variant. In one aspect, the selected bacteria or archaea can be transformed with a plasmid vector which harbors a constitutively expressed gene in coding a specific enzyme that contains an appropriate signal sequences. Alternatively, the bacterium or archaean of choice can have the enzyme gene inserted into the bacterial chromosome by transduction, linear recombination, or any other suitable method instead of using an extra chromosomal vector thereby eliminating the need for an exogenous vector.

An enzyme can be expressed by transformation of a suitable host organism, for example, by use of either prokaryotic or eukaryotic host cells. Examples of host cell types include, without limitation, bacterial cells (e.g., E. coli), yeast cells (e.g., pichia, S. cerevisiae), cultured insect cell lines (e.g., Drosophila), plant cell lines (e.g., maize, tobacco, rice, sugarcane, potato tuber), mammalian cells lines (e.g., Chinese Hamster Ovary (CHO)). In one aspect, a recombinant host cell system can be selected that processes and post-translationally modifies nascent polypeptides in a manner desired to produce the final catalytic enzyme.

A nucleic acid sequence that encodes an enzyme may be placed in an expression vector for expression in the selected host. Such expression vectors can generally include a transcriptional initiation region linked to the nucleic acid sequence that encodes the enzyme. An expression vector can also include a plurality of restriction sites for insertion of the nucleic acid to be under the transcriptional regulation of various control elements. The expression vector additionally may contain selectable marker genes. Suitable control elements such as enhancers/promoters, splice junctions, polyadenylation signals, etc. may be placed in close proximity to the coding region to permit proper initiation of transcription and/or correct processing of the primary transcript, i.e., the coding region for the enzyme. Alternatively, the coding region utilized in an expression vector may contain endogenous enhancers/promoters, splice junctions, intervening sequences, polyadenylation signals, etc., or a combination of both endogenous and exogenous control elements.

An expression vector generally includes in the 5′-3′ direction of transcription, a promoter, a transcriptional and translational initiation region, a DNA sequence that encodes the enzyme, and a transcriptional and translational termination region functional in the host cell. In one aspect, a T7-based vector can be used, which can include at least the following components: an origin of replication, a selectable antibiotic resistance gene (e.g.-ampr, tetr, chlrr), a multiple cloning site, T7 initiator and terminator sequences, a ribosomal binding site, and a T7 promoter.

In general, any suitable promoter may be used that is capable of operative linkage to the heterologous DNA such that transcription of the DNA may be initiated from the promoter by an RNA polymerase that may specifically recognize, bind to, and transcribe the DNA in an open reading frame. Some useful promoters include, constitutive promoters, inducible promoters, regulated promoters, cell specific promoters, viral promoters, and synthetic promoters. Moreover, while promoters may include sequences to which an RNA polymerase binds, this is not a requirement. A promoter may be obtained from a variety of different sources. For example, a promoter may be derived entirely from a native gene of the host cell, be composed of different elements derived from different promoters found in nature, or be composed of nucleic acid sequences that are entirely synthetic. A promoter may be derived from many different types of organisms and tailored for use within a given cell. For example, a promoter may include regions to which other regulatory proteins may bind in addition to regions involved in the control of the protein translation, including coding sequences.

A translation initiation sequence can be derived from any source, e.g., any expressed E. coli gene. Generally, the gene is a highly expressed gene. A translation initiation sequence can be obtained via standard recombinant methods, synthetic techniques, purification techniques, or combinations thereof, which are all well known. Alternatively, translational start sequences can be obtained from numerous commercial vendors. (Operon Technologies; Life Technologies Inc.).

The termination region may be native with the transcriptional initiation region, may be native with the coding region, or may be derived from another source. Transcription termination sequences recognized by the transformed cell are regulatory regions located 3′ to the translation stop codon, and thus together with the promoter flank the coding sequence. Examples include transcription termination sequences derived from genes with strong promoters, such as the trp gene in E. coli as well as other biosynthetic genes.

Vectors that may be used include, but are not limited to, those able to be replicated in prokaryotes and eukaryotes. For example, vectors may be used that are replicated in bacteria, yeast, insect cells, and mammalian cells. Examples of vectors include plasmids, phagemids, bacteriophages, viruses (e.g., baculovirus), cosmids, and F-factors. Specific vectors may be used for specific cell types. Additionally, shuttle vectors may be used for cloning and replication in more than one cell type. Such shuttle vectors are known in the art. The vector may, if desired, be a bi-functional expression vector that may function in multiple hosts.

An expression vector that encodes an extremophilic enzyme, such as, for example, thermophilic enzyme, may be introduced into a host cell by any method known to one of skill in the art and the nucleic acid constructs may be carried extrachromosomally within a host cell or may be integrated into a host cell chromosome, as desired. A vector for use in a prokaryote host, such as a bacterial cell, includes a replication system allowing it to be maintained in the host for expression or for cloning and amplification. A vector may be present in the cell in either high or low copy number. Generally, about 5 to about 200, and usually about 10 to about 150 copies of a high copy number vector are present within a host cell. A host cell containing a high copy number vector will preferably contain at least about 10, and more preferably at least about 20 plasmid vectors. Generally, about 1 to 10, and usually about 1 to 4 copies of a low copy number vector will be present in a host cell. In many aspects, bacteria are used as host cells. Examples of bacteria include, but are not limited to, Gram-negative and Gram-positive organisms. In one aspect an E. coli expression system suitable for T7 protein expression may be used. Examples of T7 expression strains can include, without limitation, BL21 (DE3), BL21 (DE3) pLysS, BLR (DE3) pLysS, Tuner (DE3) pLysS, Tuner (DE3),

Lemo21 (DE3), NiCO2 (DE3), Oragami2 (DE3), Origami B (DE3), Shuffle T7 Expres, HMS174 (DE3), HMS174 (DE3) pLysS, DH5aplhaE, Rosetta2 (DE3), Rosetta2 (DE3) pLysS, NovaBlue (DE3), Rosetta-gami B, Rosetta-gami B (DE3), Rosetta-gami B (DE3) pLysS, Rosetta Blue (DE3), Novagen (DE3), Novagen (DE3) pLysS.

An expression vector may be introduced into bacterial cells by commonly used transformation/infection procedures. A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrating vectors may also contain bacteriophage or transposon sequences. Extrachromosomal and integrating vectors may contain selectable markers to allow for the selection of bacterial strains that have been transformed.

Useful vectors for an E. coli expression system may contain constitutive or inducible promoters to direct expression of either fusion or non-fusion proteins. With fusion vectors, a number of amino acids are usually added to the expressed target gene sequence. Additionally, a proteolytic cleavage site may be introduced at a site between the target recombinant protein and the fusion sequence. Once the fusion protein has been purified, the cleavage site allows the target recombinant protein to be separated from the fusion sequence. Enzymes suitable for use in cleaving the proteolytic cleavage site include TEV, Factor Xa and thrombin. Fusion expression vectors which may be useful in the present can include those which express, for example and without limitation, Maltose Binding Protein (MBP), Thioredoxin (THX), Chitin Binding Domain (CBD), Hexahistadine tag (His-tag) (SEQ ID NO: 3), glutathione-S-transferase protein (GST), FLAG peptide, N-utilization substance (NusA), or Small ubiquitin modified (SUMO) fused to the target recombinant enzyme.

Methods for introducing exogenous DNA into a host cell are available in the art, and can include the transformation of bacteria treated with CaCl2 or other agents, such as divalent cations and DMSO. DNA can also be introduced into host cells by electroporation, use of a bacteriophage, ballistic transformation, calcium phosphate co-precipitation, spheroplast fusion, electroporation, treatment of the host cells with lithium acetate or by electroporation. Transformation procedures usually vary with the bacterial species to be transformed.

Following transformation or transfection of a nucleic acid into a cell, the cell may be selected for the presence of the nucleic acid through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes that render the recipient host cell resistant to drugs such as actinomycin CI, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. Selectable markers may also include biosynthetic genes, such as those in the histidine, tryptophan, and leucine biosynthetic pathways. Upon transfection or transformation of a host cell, the cell is placed into contact with an appropriate selection agent.

When modifying a microorganism, any suitable gram positive or gram negative bacteria may be used. For instance, the modified bacteria can be obtained from the genus Streptomyces. Particular examples of microorganisms from the above genus include Streptomyces thermovulgaris, Streptomyces thermoolivaceus, Streptomyces thermohygroscopicus, Streptomyces thermocarboxydovorans, or mixtures thereof.

The following genera may further be selected in accordance with the present disclosure to express enzymes of the present invention:
Firmicutes: Bacillus, Lihuaxuella, and Clostridium;
Proteobacteria: Bradyrhizobium, Sphingomonas, Azotobacter, Azospirillum, Nitrobacter, Lysobacter, Stenotrophomonas, Rhizobium, Acinetobacter, Thiobacillus, Schlegelella, Janthinobacterium, Sinorhizobium, Pseudomonas, Agrobacterium, and Escherichia (e.g. Escherichia coli);
Actinobacteria: Rhodococcus, Arthobacter, Streptomyces, Conexibacter, Rhodococcus, Solirubrobacte, Micrococcus, Rubrobacter, and Actinomyces;
Bacteroidetes: Flavobacterium and Pedobacter;
Deinococcus-thermus: Deinococcus and Thermus;
Gemmatimonadetes: Gemmatimonas and Gemmatirosa;
Spirochaetes: Tumeriella and Leptospira;
Verrucomicrobia: Pedosphaera, Chthoniobacter, and Verrucomicrobia;
Chloroflexi: Thermogemmatispora and Dictyobacter; and
Armatimonadetes: Fimbriimonas

It should be understood that the following list is exemplary only. The particular genera can be selected based on temperature, oxygen availability, salinity, other environmental characteristics, and the like.

The following organisms may further be selected in accordance with the present disclosure to express enzymes of the present disclosure (e.g., the purified enzyme): Lysobacter aestuarii, Lysobacter antibioticus, Lysobacter bugurensis, Lysobacter capsica, Lysobacter enzymogenes, Lysobacter lacus, Lysobacter lycopersici, Lysobacter maris, Lysobacter niastensis, Lysobacter profundi, Lysobacter sp., Lysobacter sp. A03, Lysobacter sp. cf310, Lysobacter sp. H21R20, Lysobacter sp. H21R4, Lysobacter sp. H23M41, Lysobacter sp. R19, Lysobacter sp. Root604, Lysobacter sp. Root690, Lysobacter sp. Root916, Lysobacter sp. Root983, Lysobacter sp. TY2-98, Lysobacter spongiae, Lysobacter spongiicola, Lysobacter, Lysobacter alkalisoli, Lysobacter arseniciresistens, Lysobacter daejeonensis, Lysobacter dokdonensis, Lysobacter enzymogenes, Lysobacter enzymogenes, Lysobacter gilvus, Lysobacter gummosus, Lysobacter maris, Lysobacter oculi, Lysobacter panacisoli, Lysobacter penaei, Lysobacter prati, Lysobacter psychrotolerans, Lysobacter pythonis, Lysobacter ruishenii, Lysobacter segetis, Lysobacter silvestris, Lysobacter silvisoli, Lysobacter soli, Lysobacter sp., Lysobacter sp. 17J7-1, Lysobacter sp. Alg18-2.2, Lysobacter sp. Cm-3-T8, Lysobacter sp. H23M47, Lysobacter sp. HDW10, Lysobacter sp. 114, Lysobacter sp. N42, Lysobacter sp. OAE881, Lysobacter sp. Root494, Lysobacter sp. URHA0019, Lysobacter sp. WF-2, Lysobacter sp. yr284, Lysobacter tabacisoli, Lysobacter telluris, Lysobacter tolerans, Lysobacter tolerans, Lysobacter xinjiangensis, unclassified Lysobacter, Aliivibrio finisterrensis, Aliivibrio fischeri, Aliivibrio sifiae, Aliivibrio sp., Alivibrio sp. 1S128, Aliivibrio sp. EL58, Aliivibrio sp. SR45-2, Caballeronia arvi, Caballeronia calidae, Caballeronia hypogeia, Caballeronia insecticola, Caballeronia pedi, Caballeronia terrestris, Dokdonella koreensis, Dyella caseinilytica, Dyella choica, Dyella dinghuensis, Dyella flava, Dyella jiangningensis, Dyella kyungheensis, Dyella mobilis, Dyella monticola, Dyella nitratireducens, Dyella psychrodurans, Dyella soli, Dyella solisilvae, Dyella sp. 7MK23, Dyella sp. ASV21, Dyella sp. ASV24, Dyella sp. C11, Dyella sp. C9, Dyella sp. DHC06, Dyella sp. EPa41, Dyella sp. G9, Dyella sp. M7H15-1, Dyella sp. M7H15-1, Dyella sp. OK004, Dyella sp. S184, Dyella sp. SG562, Dyella sp. SG609, Dyella sp. YR388, Dyella tabacisoli, Fluoribacter bozemanae, Fluoribacter dumoffii NY 23, Fluoribacter gormanii, Microscilla marina, Pseudomonas aeruginosa, Pseudomonas thermotolerans, Pseudomonas mediterranea, Psychrobacter sp., Psychromonas sp. MB-3u-54, Psychromonas sp. psych-6C06, Psychromonas sp. RZ22, Psychromonas sp. Urea-02u-13, Rhodanobacter denitrificans, Rhodanobacter fulvus, Rhodanobacter glycinis, Rhodanobacter lindaniclasticus, Rhodanobacter panaciterrae, Rhodanobacter sp. 7MK24, Rhodanobacter sp. A1T4, Rhodanobacter sp. B04, Rhodanobacter sp. B05, Rhodanobacter sp. C01, Rhodanobacter sp. C03, Rhodanobacter sp. C05, Rhodanobacter sp. C06, Rhodanobacter sp. DHB23, Rhodanobacter sp. DHG33, Rhodanobacter sp. L36, Rhodanobacter sp. MP1X3, Rhodanobacter sp. OK091, Rhodanobacter sp. OR444, Rhodanobacter sp. PCA2, Rhodanobacter sp. Root480, Rhodanobacter sp. Root627, Rhodanobacter sp. Root627, Rhodanobacter sp. SCN 67-45, Rhodanobacter sp. SCN 68-63, Rhodanobacter sp. Soil772, Rhodanobacter sp. T12-5, Rhodanobacter sp. TND4EH1, Rhodanobacter sp. TND4FH1, Rhodanobacter spathiphylli, Rhodanobacter thiooxydans, Stenotrophomonas chelatiphaga, Stenotrophomonas maltophilia, Stenotrophomonas panacihumi, Stenotrophomonas pavanii, Stenotrophomonas rhizophila, Stenotrophomonas sp. DDT-1, Stenotrophomonas sp. RIT309, Stenotrophomonas sp. SKA14, Vibrio aestuarianus, Vibrio antiquaries, Vibrio aquaticus, Vibrio tasmaniensis, Xanthomonadales bacterium, Xanthomonas albilineans, Xanthomonas arboricola, Xanthomonas axonopodis, Xanthomonas bromi, Xanthomonas campestris, Xanthomonas cannabis, Xanthomonas citri, Xanthomonas euvesicatoria, Xanthomonas fragariae, Xanthomonas hortorum,, Xanthomonas hyacinthi, Xanthomonas oryzae, Xanthomonas phaseoli, Xanthomonas pisi, Xanthomonas sacchari, Xanthomonas sp. Leaf131, Xanthomonas sp. NCPPB 1128, Xanthomonas translucens, Xanthomonas vasicola, Xanthomonas vesicatoria, or a combination thereof. It should be understood that the following list is exemplary only. The particular microorganism can be selected based on temperature, oxygen availability, salinity, other environmental characteristics, and the like.

In yet another aspect, the method disclosed herein can include enzymes that co-function effectively in the same environment characterized by the same or similar pH and temperature. For example, more than one enzyme that functions well in, for example, temperature range of about 60° C. to about 100° C. and a pH range of about 5-7 can be selected, or any suitable temperature and pH combination thereof.

In some aspects, fungal enzyme(s) may be utilized for the process of the present disclosure. For example, a fungal long-chain fatty alcohol dehydrogenase can be used which the inventors have found can greatly accelerate the reaction, although not in a thermophilic process. However, such non-thermophilic enzymes can be added to the reaction(s) of the present disclosure after a temperature is reduced.

Other fungal genera sources for enzymes that can be utilized in the present disclosure include, but is not limited to: Aureobasidium, Macroventuria, Lophium, Tothia, Trichodelitschia, Westerdykella, Didymosphaeria, Viridothelium, Delitschia, Zopfia, Myriangium, Rhizodiscina, Saccharata, Aaosphaeria, Amniculicola, Byssothecium, Aspergillus, Meira, Dissoconium, Lizonia, Aureobasidium, Morchella, Sodiomyces, Tilletiopsis, Jaminaea, Ceraceosorus, Testicularia, Tilletiopsis, Violaceomyces, Rhizopus, Altemaria, Hesseltinella, Neurospora, Ramularia, and Rhynchosporium.

Regardless of the enzymes selected, as discussed above, the bioplastic polymer produced in the disclosed process is a polyalkanoate. For example, the polyalkanoate is a polyhydroxyalkanoate or a polyhydroxybutyrate. The polyhydroxyalkanoate produced can be characterized as capable of processing in chemical reactions in a manner comparable to that of polypropylene or polyethylene. The polyhydroxyalkanoate can include any one or more of the following carbon chain lengths: C6, C7, C8, C9, C10, C11, C12, C13, C14, C15, C17, C18, C19, C20, C21, C22, C23, C24, C25, C26, C27, C28, C29, and C30.

In one aspect, PHA monomer or polymer can have a linear carbon chain. In another aspect, the PHA monomer and/or polymer can be at least about 80% homogeneous such as at least about 85% homogeneous, at least about 90% homogeneous, at least about 95% homogeneous, or at least about 99% homogeneous. However, in one aspect, it should be understood that the produced PHA monomer and/or polymer chain lengths are directly correlated with the chain lengths of the long-chain fatty acid or precursor thereof.

In another aspect, PHA monomer and/or polymer can have a mass yield that is directly proportional to an optical density measurement obtained. For example, the higher the optical density reading, the higher mass yield of the PHA monomer and/or polymer produced. In yet another aspect, the PHA monomer and/or polymer can contain substantially minimal amounts of any microplastics and/or nanoplastics. For example, contain substantially minimal amounts of any microplastics and/or nanoplastics can include microplastics and/or nanoplastics in amounts of about 0.01% to about 10% of the total mass yield, such as about 0.1% to about 9% of the total mass yield, about 1% to about 8% of the total mass yield, about 2% to about 7% of the total mass yield, about 3% to about 6% of the total mass yield, or about 4% to about 5% of the total mass yield. In other aspects, the bioplastic polymer produced in step d) can be substantially absent of any microplastics and/or nanoplastics. For example, any microplastics and/or nanoplastics, if present, may be in the PHA monomer and/or polymer produced in undetectable amounts.

In certain aspects, the PHA monomer and/or polymer can be polyhydroxyalkanoate homopolymers that include poly 3-hydroxyalkanoates (e.g., poly 3-hydroxypropionate (PHP), poly 3-hydroxybutyrate (PHB), poly 3-hydroxyvalerate (PHV), poly 3-hydroxyhexonoate (PHH), poly 3-hydroxyoctanoate (PHO), poly 3-hydroxydecanoate (PHD), and poly 3-hydroxy-5-phenylvalerate (PHPV)), poly 4-hydroxyalkanoates (e.g., poly 4-hydroxybutyrate (PHB) and poly 4-hydroxyvalerate (hereinafter referred to as PHV)), or poly 5-hydroxyalkanoates (e.g., poly 5-hydroxyvalerate (hereinafter referred to as PHV)).

In certain aspects, the PHA can be a copolymer (containing two or more different monomer units) in which the different monomers are randomly distributed in the polymer chain. Examples of PHA copolymers can include poly 3-hydroxybutyrate-co-3-hydroxypropionate (hereinafter referred to as PHB3HP), poly 3-hydroxybutyrate-co-4-hydroxybutyrate (hereinafter referred to as P3HB4HB), poly 3-hydroxybutyrate-co-4-hydroxyvalerate (hereinafter referred to as PHB4HV), poly 3-hydroxybutyrate-co-3-hydroxyvalerate (hereinafter referred to as PHB3HV), poly 3-hydroxybutyrate-co-3-hydroxyhexanoate (hereinafter referred to as PHB3HH) and poly 3-hydroxybutyrate-co-5-hydroxyvalerate (hereinafter referred to as PHB5HV), and the like, having central carbon chain lengths of up to C30 as discussed above.

In yet other aspects, the present disclosure is directed to an organism-free process for producing PHA monomers and/or polymers from long-chain fatty acids or precursors thereof. For example, the long-chain fatty acid or precursor thereof can be contacted in vitro with a purified enzyme or a mixture of purified enzymes to produce a PHA monomer and/or polymer. For example, the enzymatic reaction of the organism-free process can be carried out in a single vessel or in more than one vessel. Further the organism-free process can be performed at a temperature range from about 40° C. to about 80° C. or any variation thereof as described herein.

In one aspect, the organism-free process can include one or more further steps of contacting the long-chain fatty acid or precursor thereof in vitro with the enzyme or the mixture of enzymes by repeating the enzymatic step for each of the one or more further steps. For example, the one or more further steps can be repeated at least three times. The organism-free process can include contacting the long-chain fatty acid or precursor thereof in vitro with a first enzyme followed by one or more subsequent enzymes in a stepwise manner or the organism-free process can include contacting the long-chain fatty acid or precursor thereof in vitro with two or more enzymes simultaneously. For instance, the one or more subsequent enzymes can include at least one additional enzyme that is different than the first enzyme. Of course, as noted above, in one aspect, it should be understood that the present disclosure also includes an organism-free process of contacting the long-chain fatty acid or precursor thereof with all necessary enzymes simultaneously. However, in such an aspect, one or more of the enzymes can be repeatedly added if necessary to refresh one or more of the simultaneously added enzymes.

In another aspect, the PHA monomer and/or polymer produced in the organism-free process can include a polyalkanoate. For example, the polyalkanoate can be a polyhydroxyalkanoate and/or a polyhydroxybutyrate. The polyhydroxyalkanoate can be characterized as processing in chemical reactions in a manner comparable to that of polypropylene or polyethylene. The polyhydroxyalkanoate can have any one or more of the following carbon chain lengths: C6, C7, C8, C9, C10, C11, C12, C13, C14, C15, C17, C18, C19, C20, C21, C22, C23, C24, C25, C26, C27, C28, C29, and C30.

In one aspect, the PHA monomer and/or polymer produced in the organism-free process can include a linear carbon chain. In one aspect, the PHA monomer and/or polymer produced in the organism-free process can be at least 80% homogeneous, or any variation thereof as discuss herein. In another aspect, the PHA monomer and/or polymer produced in in the organism-free process can have a mass yield that is directly proportional to an optical density measurement obtained. In yet another aspect, the PHA monomer and/or polymer produced in in the organism-free process can contain substantially minimal amounts of any microplastics and/or nanoplastics.

In one aspect, the enzyme or the mixtures of enzymes in the organism-free process can be purified from an extremophilic microorganism as described herein. The microorganism from which the enzyme can be purified can be an engineered microorganism that has been genetically modified to secrete a specific enzyme for use the organism-free process or the microorganism from which the enzyme can be purified can be at least one type of a naturally occurring microorganism that naturally encodes a specific enzyme. For example, the enzyme or the mixtures of enzymes can be a thermophilic enzyme. The thermophilic enzyme can be temperature tolerant from about 40° C. to about 120° C. or any variation thereof as discussed herein. For instance, at least one enzyme can be purified from Lihuaxuella thermophila. In one aspect, the mixture of enzymes used can include enzymes that co-function effectively in the same environment characterized by the same or similar pH and temperature, as described herein.

In another aspect, the organism-free process can include contacting one or more long-chain fatty acid or precursor thereof with a purified enzyme or a mixture of purified enzymes in an environment substantially absent any bacteria that secrete the same enzyme or the mixture of the same enzymes to produce a linear chain PHA monomer and/or polymer. For example, the linear chain PHA monomer and/or polymer can include a polyhydroxyalkanoate having a carbon chain length greater than C8 and/or a carbon chain length less than C30.

In yet another aspect, generally, the present disclosure is directed to an uncharacterized polyhydroxyalkanoate. For example, the uncharacterized polyhydroxyalkanoate can have a carbon chain length greater than C8 and can have a linear chain polymer substantially absent any side chain pendant polymers. For example, the uncharacterized polyhydroxyalkanoate can have a carbon chain length greater than C8 but less than C30 and can have a linear chain polymer.

In another aspect, the present disclosure is generally directed to a process of producing a polyalkenoate in a multi-step enzymatic reaction. For example, the process can include contacting a long-chain fatty acid (if precursor thereof, contacted with the necessary enzymes to yield the long-chain fatty acid), contacting the long-chain fatty acid with a long chain fatty acid CoA ligase/synthetase to obtain a long-chain fatty acid acyl-CoA, contacting the long-chain fatty acid acyl-CoA with a long chain acyl-CoA dehydrogenase, followed by a long-chain-enoyl-CoA hydratase, followed by a hydroxyacyl-CoA dehydrogenase to obtain a long-chain acetoacetyl-CaA, and contacting a long-chain acetoacetyl-CaA with a hydroxyacyl coenzyme-A dehydrogenase to obtain a hydroxyacyl-CoA for polymerization into a polyhydroxyalkanoate or contacting a long-chain acetoacetyl-CaA with an acetyl-CoA C-acyltransferase to obtain acetyl-CoA and contacting the acetyl-CoA with an acetoacetyl-CoA synthase to obtain a polyhydroxyalkanoate (such as PHB in this example). Of course, as noted above, in one aspect, each of the above referenced enzymes can be added simultaneously. Namely, as discussed, by carefully selecting compatible enzymes, such as those discussed above, a one-pot reaction is possible, allowing all enzymes to be added simultaneously such that the reaction proceeds naturally from the long-chain fatty acid or precursor thereof to the production of a PHA without further intervention. However, as discussed, in one aspect, one or more of the simultaneously added enzymes can be added or “refreshed” during the one-pot process.

In some aspect, the long chain fatty acid CoA ligase/synthetase is purified from Thermobifida halotolerans or any other microorganism described herein. For example, the alcohol dehydrogenase can a fungal long-chain alcohol purified dehydrogenase as described herein.

The present disclosure may be better understood with reference to the following example.

### EXAMPLES

The present disclosure may be better understood with reference to the following examples.

### Materials and Methods

Sources: Enzyme sequences were identified by searching the NCBI Protein or Identical Protein Group databases. For this example, it was decided to focus primarily on the thermohalophile Thermobifida halotolerans. Sequences and accession numbers for the enzymes used in this work are listed in Appendix I. All general laboratory chemicals and long chain fatty acids were purchased from Millipore-Sigma Chemical Company (St. Louis, MO). Cloning materials and protein purification supplies were from New England Biolabs (Beverly, MA).
Enzyme cloning: The amino acid sequence for each enzyme was searched for potential signal sequences. If one was present it was removed from the sequence. A histine tag and a TEV protease cleavage sequence were added to the N-ter of each enzyme. This sequence was MHHHHHHENLYFQS to assist in purification (via an NTA-Ni column) and subsequent removal of the hexahistidine tag. Each recombinant enzyme began with an N-ter serine residue as TEV protease cleaves after the glutamine (Q). Each amino acid sequence was back translated to an E. coli codon optimized nucleic acid sequence and was synthesized using standard laboratory gene synthesis techniques. The insert was digested with Nde I and cloned into the similarly digested expression vector pET11d. Final DNA sequences were verified by DNA sequencing. Expression plasmids were used to transform E. coli Origami2 (DE3) for expression.
Protein expression and purification: A single colony was used to inoculate 5.0 mL of LBₐₘₚ media (80 mg/ml ampicillin concentration). Bacteria were grown for 12 hours at 37° C. and this culture was used to inoculate 500 mL of LBₐₘₚ media. This culture was grown at 37° C. until the optical density (600 nm) reached 0.4 (typically in 3-5 hours). Protein expression was induced by the addition of IPTG to a final concentration of 1 mM. Cells were grown for 12 hours at 37° C. Cells were harvested by centrifugation at 5,000×g for 20 minutes, and frozen at −80° C. until use (minimal time frozen was 24 hours). Cells were thawed on ice and were resuspended in Buffer A (0.5 M NaCl, 20 mM Tris-HCl, 5 mM imidazole, pH 7.9) (typically 1 mL per gram of cells). Cells were disrupted via two passes through a French Press followed by centrifugation at 30,000×g for 30 minutes. The crude extract was mixed with an equal volume of charged His-Bind resin slurry and the mixture was poured into 5 cm×4.9 cc column. The column was washed with 10 column volumes of wash buffer (0.5 M NaCl, 20 mM Tris-HCl, 60 mM imidazole, pH 7.9) at a flow rate of 0.2 mL/min. Enzyme was eluted from the column with the addition of 3 column volumes of 0.5 M NaCl, 20 mM Tris-HCl, 1.0 M imidazole, pH 7.9. Fractions were collected (1.0 mL). Fractions containing enzyme were pooled after analysis by SDS PAGE. The pooled fractions were applied to a 70 cmp×4.9 cc Sephadex G-75 column (10 mM Tris-HCl, PH 7.5, 1 mM EDTA). Fractions containing homogeneous protein were pooled (after inspection by SDS PAGE) and concentrated to 5 mg/ml via Centricon filters. Enzyme was stored frozen at −20° C. until use. The TRX-histidine tag region was removed from the enzymes using TEV protease. Protein was diluted to 1.0 mg/ml into 10 mM Tris-HCl, pH 7.5, 25 mM NaCl. 100 U of TEV protease was added per mg of enzyme (approximate ratio of 1:100 (w/w). The reaction was allowed to proceed for 16 h at 4° C. The mixture was passed over a charged nickel column. One column volume of eluent was collected representing purified tag-free enzyme.
Enzyme assays: The formation of long-chain fatty acid-CoA was monitored by measuring the amount of pyrophosphate released from the reaction using the MAK 168 pyrophosphate assay kit from Millipore-Sigma Chemical Co (St. Louis, MO). The formation of PHA was monitored by the increase in optical density (650 nm) as a function of time in a Molecular Devices SpectraMax 5 spectrometer. The assay measures the increase in light scattering due to precipitating PHA formed during the reaction. The depolymerization of isolated PHA material was performed using the Lihuaxuella thermophilia PHB depolymerase that was previously characterized (Quirk, 2020. TL-23500). Standard reactions conditions were 10 mM KCl, 10 mM MgCl₂, 20 mM CHES (pH 9.0), 55° C.

A turbidometric assay was employed to measure PHBDase activity under various conditions. The standard reaction (final volume=1.0 mL) contained 200 mg/L of PHB granules (that were previously stably suspended via sonication), 1 mM CaCl2, 10 mM KCl, 0.5 M NaCl, 20 mM buffer at various pH values. The reaction was initiated after the addition of enzyme and monitored at 650 nm in Applied Photophysics spectrapolarometer in absorbance mode. The reaction was gently stirred and maintained at a constant temperature. OD measurements (typically starting in the range of 2-3) were converted to percent OD remaining as a function of time. All reported kinetic constants are per Segel (1993).

The reduction in the amount of fatty acid in the initial reactions was measured using the Millipore-Sigma Chemical Co. Fatty Acid Quantitation kit. During the initial reaction, aliquots were removed as a function of time and the remaining fatty acid content was measured with the kit. Fluorescence emission intensity was measured at 587 nm (excitation wavelength 535 nm) on a Molecular Devices SpectraMax 5 spectrometer.

### EXAMPLE 1

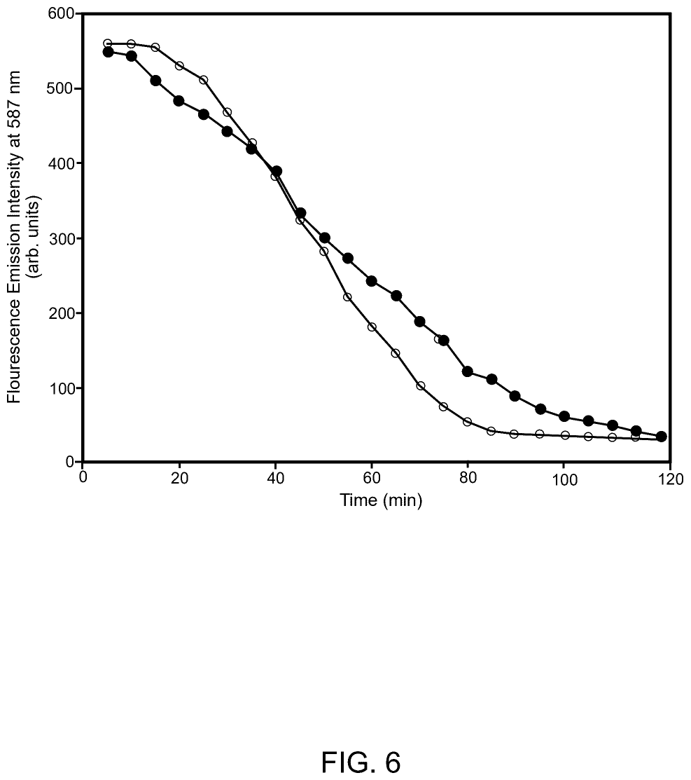

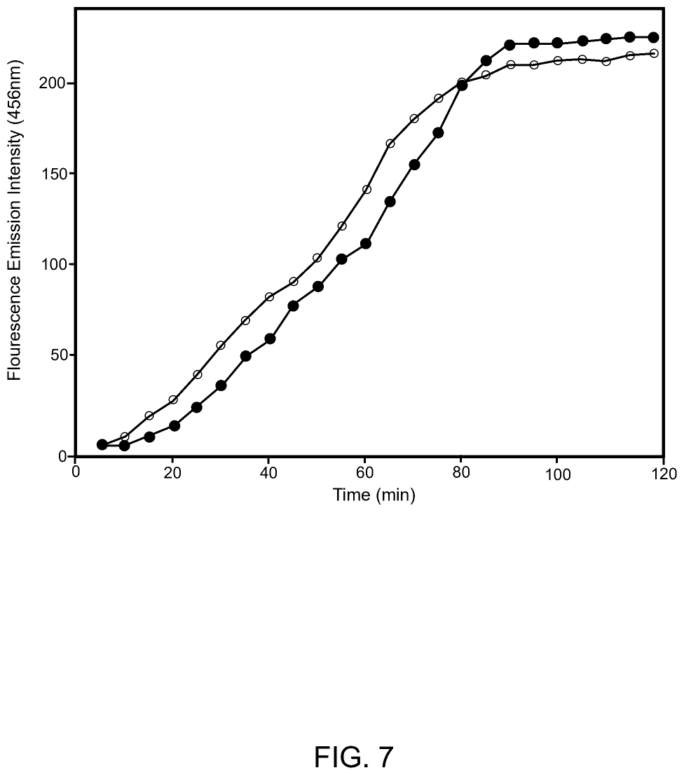

Using the enzymatic assay for fatty acids, the activity of the long-chain fatty acid CoA ligase reaction for two different fatty acids (the reaction shown in FIG. 2) was measured. The time course of these reactions is shown in FIG. 6. Shown in the graph is the conversion for arachidic acid (C18) and myristic acid (C12). Although the kinetics of the reaction are slightly different over the course of the assay, both reactions are complete within two hours under the conditions tested. Coincident with the loss of measurable fatty acid in the reaction is the formation of the pyrophosphate that is liberated in the formation of long-chain fatty acid CoA molecules. The time course of the reaction completely mirrors the loss of fatty acid. This is shown in FIG. 7. Hence the key reaction in this disclosure has been clearly demonstrated for two mid-length fatty acids.

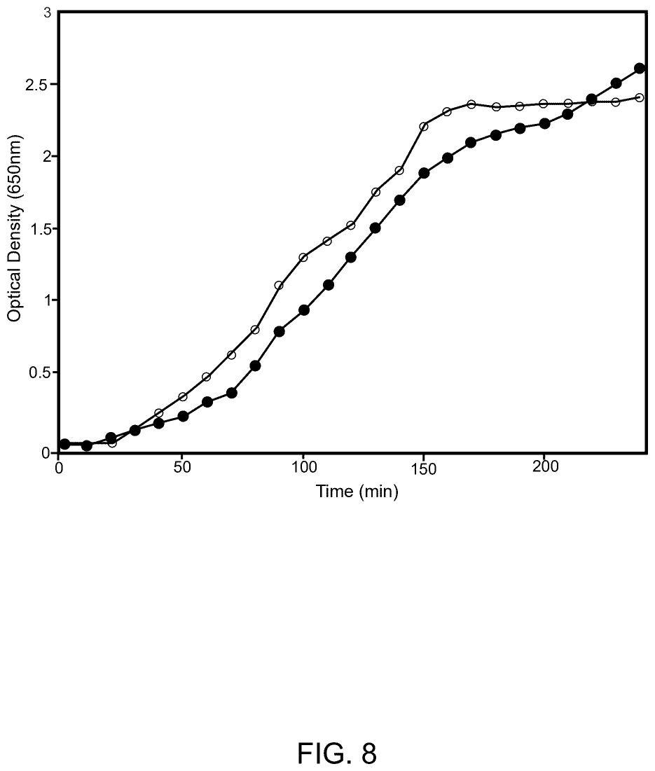

Once the long-chain fatty acid CoA moiety is produced, it was used as the input to the reaction shown in FIG. 4. The overall 5-step reaction is easily monitored by the formation of the PHA that precipitates from solution and alleviates the need to follow the course of any or all of the other enzymatic steps. FIG. 8 shows that there is a measurable increase in optical density due to light scattering (650 nm) after a lag of approximately 50 minutes. This lag corresponds to the time it takes for the first four enzymatic steps to be accomplished and for sufficient substrate to accumulate for the final reaction to take place. The formation of precipitating PHA is then linear for the next 150 minutes, followed by the end of the reaction for arachidic acid (as starting material) or a biphasic slowing of the reaction when myristic acid is used as the beginning substrate. Although the slopes of the linear portion of the reaction are nearly identical, it is not clear what is driving the behavior at the end of the assays. It could be that substrate deletion is beginning to affect (in a kcat/Km manner) the reaction that utilizes arachidic acid. The main observation is that utilizing this scheme it is possible to efficiently produce precipitating material.

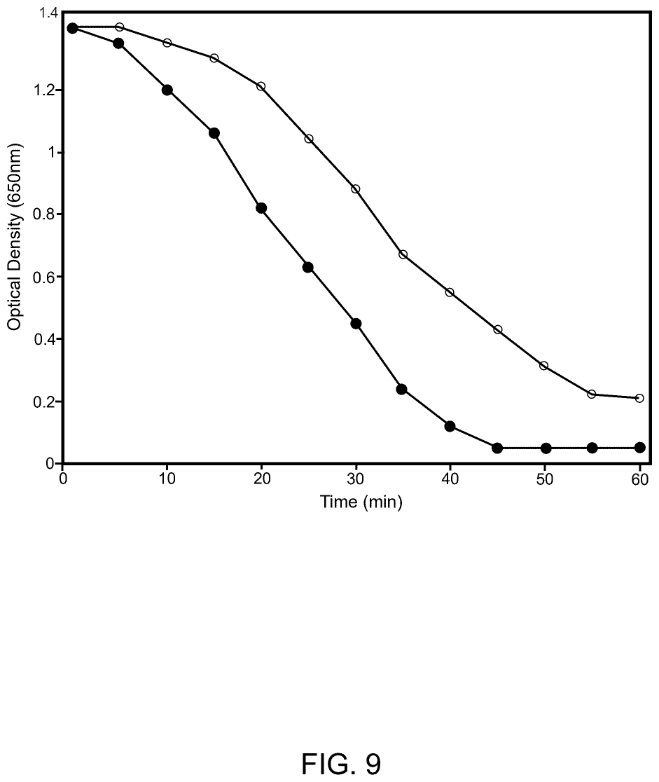

The precipitated PHA material was separated from reaction components by brief centrifugation and two rounds of washing in phosphate buffered saline. The final pelleted material was resuspended in depolymerase reaction buffer (see Methods) and was mixed with the PHB depolymerase from Lihuaxuella thermophilia. As is shown in FIG. 9, the PHA material formed using either arachidic or myristic acid can be depolymerized over the course of an hour as measured by the loss of light scattering. The myristic acid-based reaction can be driven to near completion (as evidenced by near zero optical density at 60 minutes) whereas the arachidic acid-based reaction ends with some undegraded material remaining.

### EXAMPLE 2

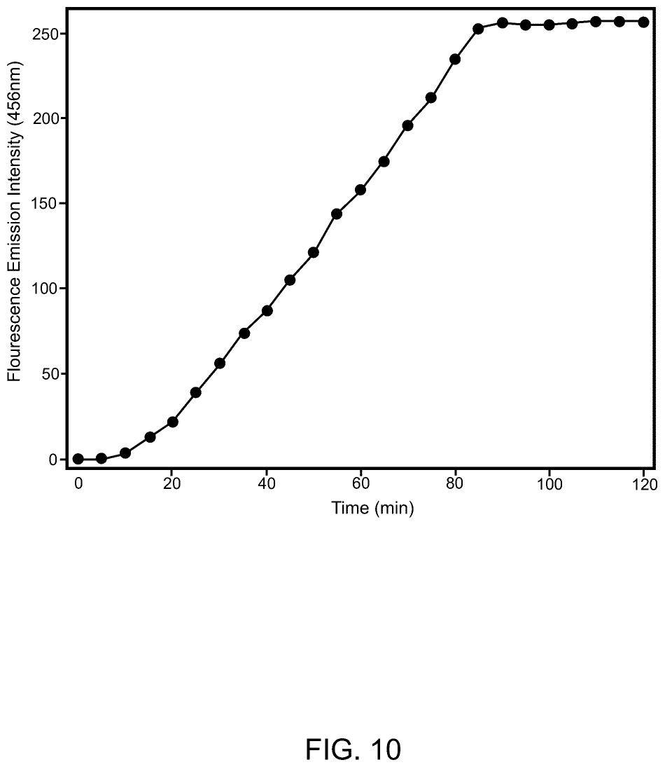

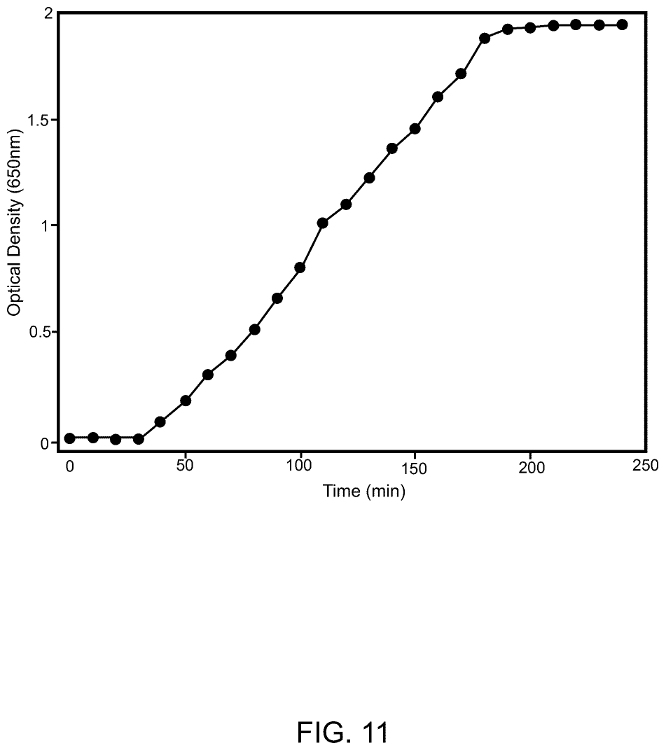

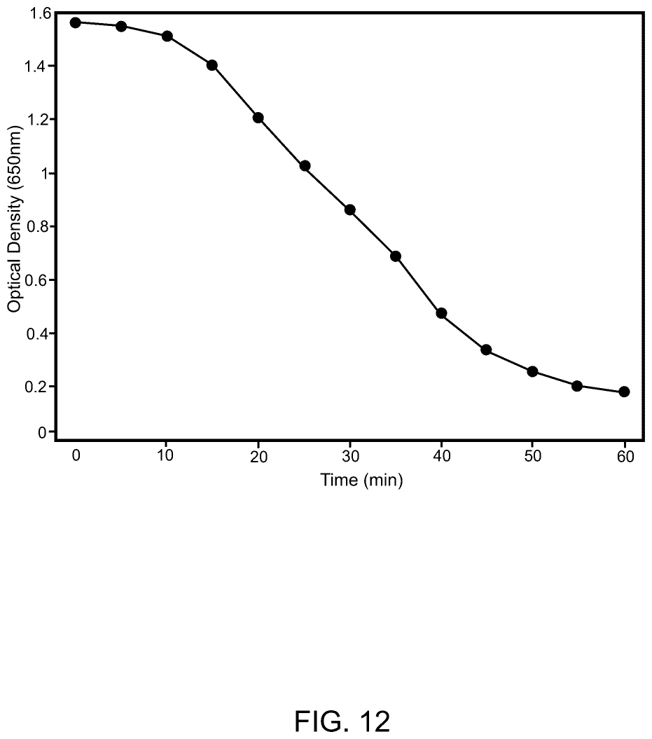

A homogeneous chain length PHA was formed from two single chain length fatty acids following the overall enzymatic steps shown in FIGS. 2 and 3. Namely, an equimolar amount of arachidic and myristic acids was fully converted into their respective CoA moieties using the WP_068688876 enzyme. This is shown in FIG. 10 by following the formation of pyrophosphate. The time course indicates that the reaction is completed within 90 minutes. The pool of the two CoA moieties was then used as the substrate for the formation of a mixed C12/C18 PHA polymer as shown by the increase in light scattering as material precipitates from the overall reaction as a function of time (FIG. 11). After the overall reaction was completed, the isolated mixed C12/C18 PHA polymer was subjected to depolymerization using the L. thermophilia PHA depolymerase as is shown in FIG. 12. The reaction is characterized by a short lag phase (approximately 10 minutes) followed by robust depolymerization and a trailing off the reaction rate after 55 minutes. After an hour approximately 12% of the input mixed polymer remains. This most likely reflects substrate preference of the single depolymerase used in this reaction. Still, it does confirm that polymer is formed in the mixed substrate scenario that can be depolymerized by a known PHA depolymerase. As illustrated, it is possible to mix and match substrates to create novel PHAs that are multiples of that input. In this case C12/C18. By varying the substrate input ratios it will be possible to create novel PHAs with compositions that reflect those ratios.

### EXAMPLE 3

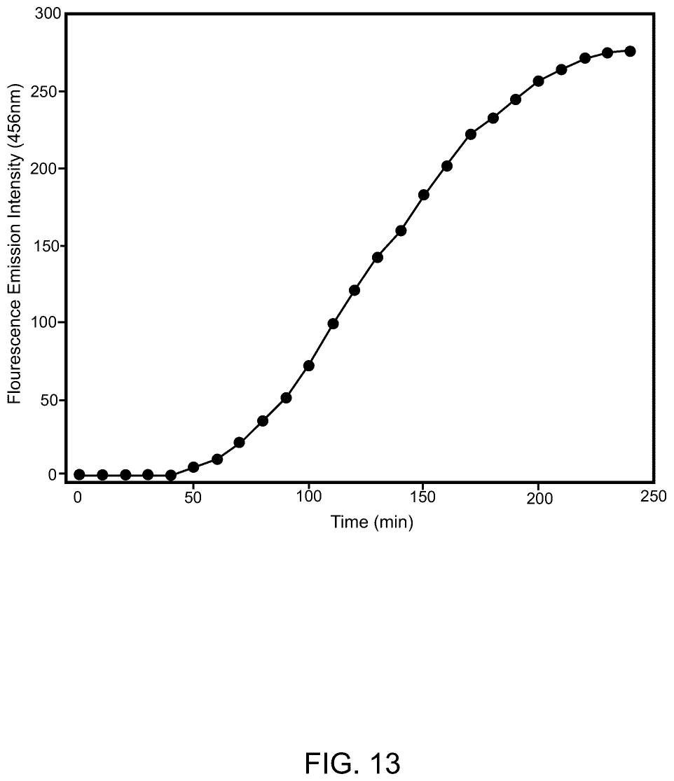

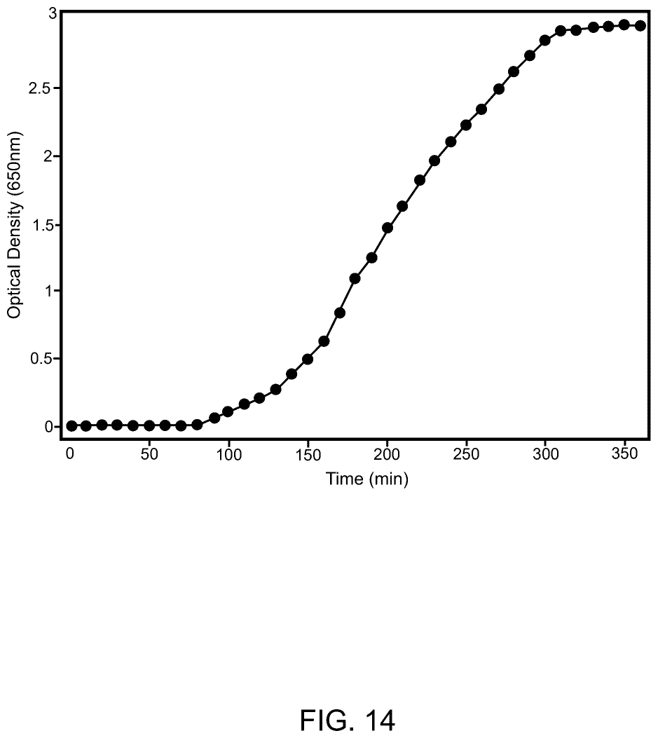

Arachidic and myristic acids represent approximately the mid-range of available fatty acids (see, e.g. FIG. 3). To test if a broader range of chain lengths could be utilized, the formation of PHA was conducted using the C24 fatty acid hexacosanoic acid. The formation of the CoA moiety using the WP_068689753 enzyme is significantly slower than the other reactions (FIG. 13); marked by a 50-minute lag period and completion only after 240 minutes. However, the reaction sill proceeded, and the formed CoA moiety was able to be converted into measurable and precipitating polymer as is seen in FIG. 14.

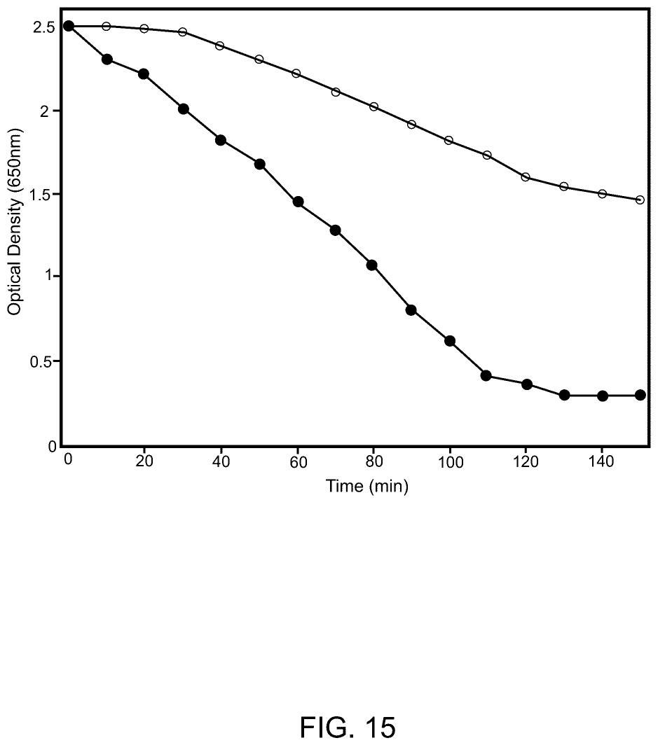

Interestingly, the hexacosanoic acid-derived polymer displays differential depolymerization when two different PHA polymerases are employed in the depolymerase reaction. The depolymerase from Thermus thermophilus is significantly more capable of depolymerizing the polymer than is the enzyme from Lihuaxuella thermophilia as is shown in FIG. 15. The polymer can be degraded by both enzymes, but at different rates and extents. Both enzymes leave unreacted polymer and/or insoluble reaction by-products. Thus it is of note that just as choice of enzyme for the formation of long-chain fatty acid CoA molecules, it is important to use an ideal/compatible depolymerase as part of further polymer studies.

### EXAMPLE 4

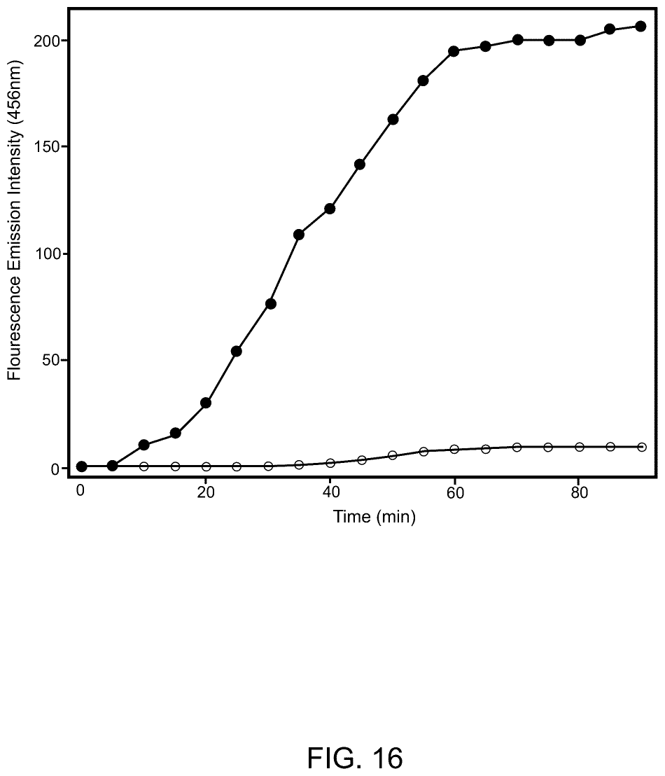

Smaller chain length fatty acids were also utilized to create novel PHAs. Heptanoic acid (C5) was readily converted into the CoA moiety when utilizing the WP_068692787 enzyme. The reactions for heptanoic acid are shown in FIG. 16, namely, which illustrates the formation of the pyrophosphate in the initial reaction of heptananoic acid.

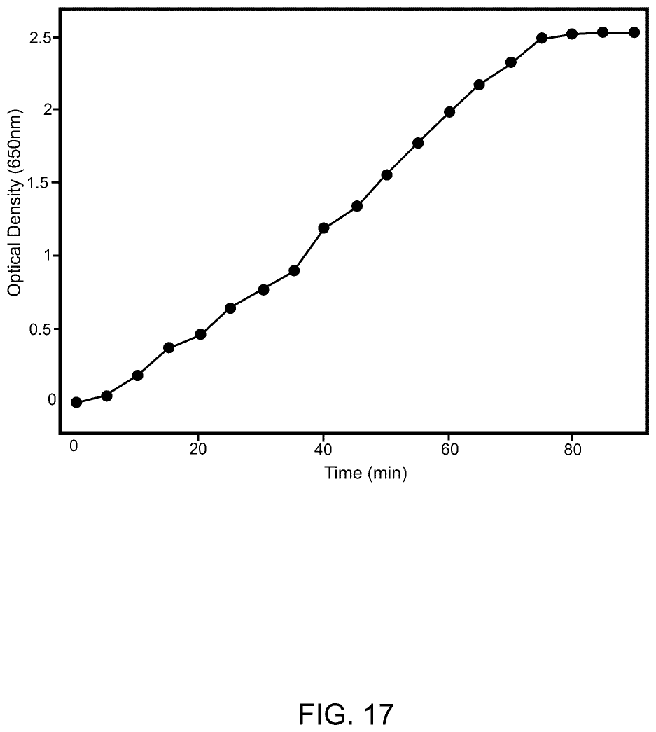

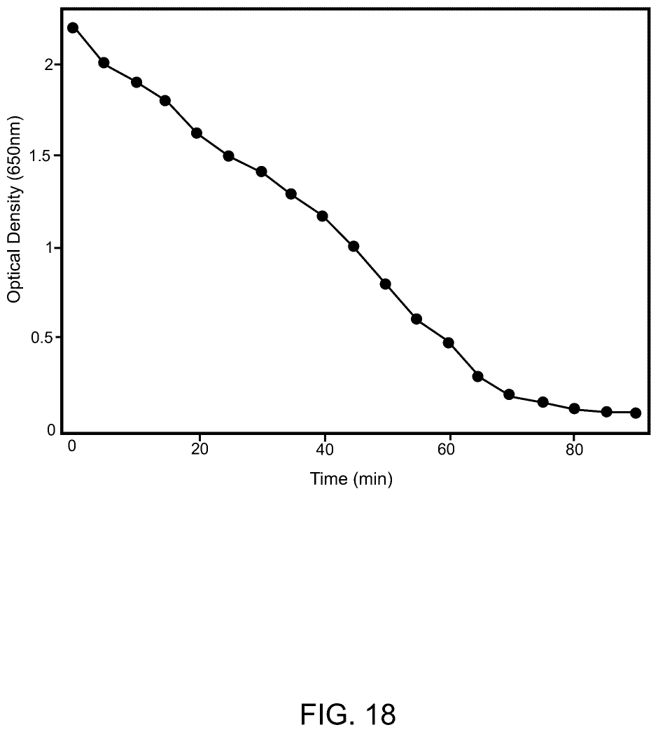

Using the optical density assay (FIG. 17) of the formation of PHA and the heptanoic-CoA substrate, it was illustrated that the PHA was formed, which precipitates out of solution as a function of time. This reaction is shown in FIG. 17. Similarly, the PHA was rapidly depolymerized using the L. thermophilia PHB depolymerase as is seen in FIG. 18.

### EXAMPLE 5

Novel homogenous PHA polymers and copolymers were formed using various enzymes isolated from Halomonas titanicae and three fatty acid starting substrates with varying chain lengths: octanoic acid (C6), myristic acid (C12), and behenic acid (C20). Enzymes were added to a final concentration of 20 μg/mL and fatty acid starting substrates were added to a final concentration of 10 mg/mL. Reaction buffer contained 10 mM Tris-HCl (pH 7.5), 5 mM ATP, 10 mM MgCl₂, 10 mM KCl, 10 mM MnCl₂, 10 mM coenzyme A, 10 mM flavin adenine dinucleotide, 1.0 M NaCl, 5 μg/mL Halomonas titanicae inorganic pyrophosphatase (E.C. 3.6.1.1), 10 mM NADH, 20 μg/mL polyphosphate: AMP phosphotransferase (E.C. 2.7.4.3x, Halomonas halophila), and 10 mg/mL linear polyphosphate, isocitrate dehydrogenase (E.C. 1.1.1.41/42, Halomonas titanicae), and 100 mM isocitrate.

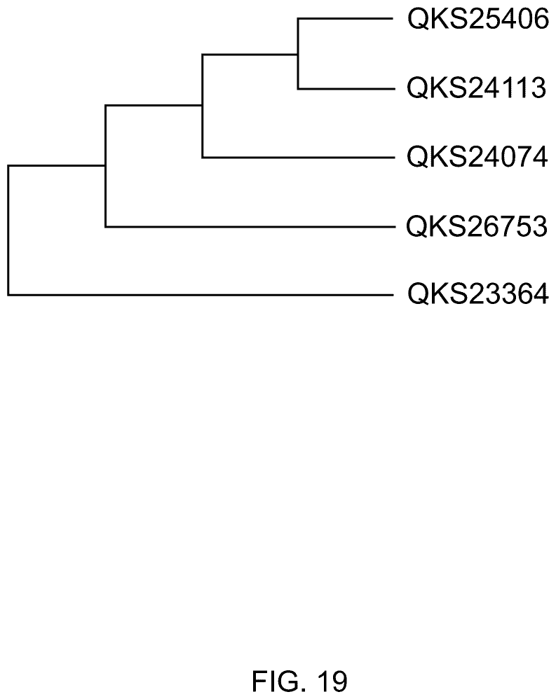

First, the long-chain fatty acids were converted to long-chain fatty acid-CoA molecules (the reaction shown in FIG. 2) using ligase enzymes isolated from Halomonas titanicae. Halomonas titanicae expresses ten fatty acid-CoA ligases, several of which are nearly identical. A percent identity matrix of the remaining five most unique fatty acid-CoA ligases expressed by Halomonas titanicae (QKS24074, QKS24113, QKS25406, QKS26753, and QKS23364) showed that the five most unique ligases were between 22 and 32 percent identical (Table I). QKS24074 and QKS24113 were the most distinct enzymes at 22.18 percent identical, and QKS26753 and QKS23364 were the most similar enzymes at 32.99 percent identical. FIG. 19 is a consensus maximum likelihood tree, which further shows the relatedness between the five most unique fatty acid-CoA ligases expressed by Halomonas titanicae. The plethora of enzymes available indicates that care must be taken in selecting the appropriate enzyme to match the long-chain fatty acid chain length.

TABLE I
Percent Identity Matrix-Fatty acid CoA ligases.

| | | 1 | 2 | 3 | 4 | 5 |
|---|---|---|---|---|---|---|
| 1: | QKS24074 | 100.00 | 22.18 | 25.76 | 26.12 | 28.40 |
| 2: | QKS24113 | 22.18 | 100.00 | 30.54 | 27.18 | 27.52 |
| 3: | QKS25406 | 25.76 | 30.54 | 100.00 | 30.24 | 31.12 |
| 4: | QKS26753 | 26.12 | 27.18 | 30.24 | 100.00 | 32.99 |
| 5: | QKS23364 | 28.40 | 27.52 | 31.12 | 32.99 | 100.00 |

While it would have been possible to run the reaction shown in FIG. 2 with a choice of enzymes, optimal enzymes were selected for each of the three chain length fatty acids from the five Halomonas titanicae fatty acid-CoA ligase enzymes: QKS24074, QKS24113, QKS25406, QKS26753, and QKS23364. The substrate chain length preference distribution (percent activity vs. carbon length) for each of the five fatty acid-CoA ligases is shown in Table II. For octanoic acid (C6), the results showed that QKS24074 was the optimal enzyme; QKS23364 was a possible enzyme but had much lower activity; and QKS25406, QKS24113, and QKS26753 were not possible enzymes. For myristic acid (C12), the results showed that QKS26753 or QKS23364 were the optimal enzymes and QKS25406, QKS24113, or QKS24074 were possible but had much lower activity. For behenic acid (C20), the results showed that QKS25406 or QKS24113 were the optimal enzymes, QKS23364 was a possible enzyme but had much lower activity, and QKS24074 and QKS26753 were not possible enzymes. The inventors found that while it was possible to run the reaction with enzymes having vastly lower activities against the fatty acid, it was much less preferred. The inventors also found that it was possible to run the reaction in the presence of two moieties with different chain lengths and with one or more than one fatty acid-CoA ligase enzyme.

| Chain | | | | | |
|---|---|---|---|---|---|
| Length | QKS25406 | QKS24113 | QKS24074 | QKS26753 | QKS23364 |
| C6 | 0.0 | 0.0 | 100 | 0.0 | 25.0 |
| C8 | 0.0 | 0.0 | 100 | 0.0 | 70.7 |
| C10 | 0.0 | 0.0 | 83.3 | 66.6 | 100 |
| C12 | 11.6 | 5.2 | 21.0 | 100 | 100 |
| C14 | 30.4 | 21.1 | 0.0 | 100 | 100 |
| C16 | 100 | 97.5 | 0.0 | 42.4 | 100 |
| C18 | 100 | 100 | 0.0 | 5.6 | 100 |
| C20 | 100 | 100 | 0.0 | 0.0 | 46.5 |
| C22 | 89.9 | 100 | 0.0 | 0.0 | 20.2 |

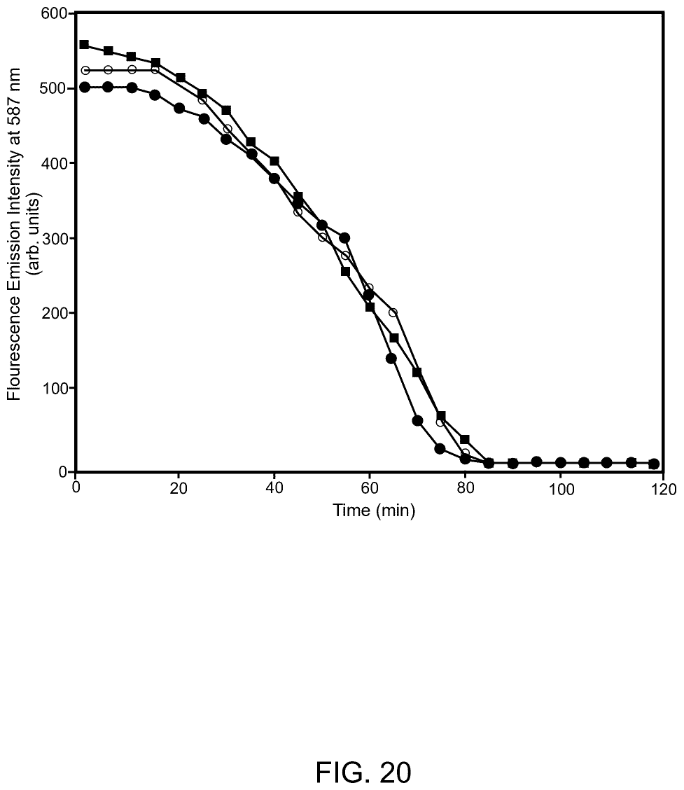

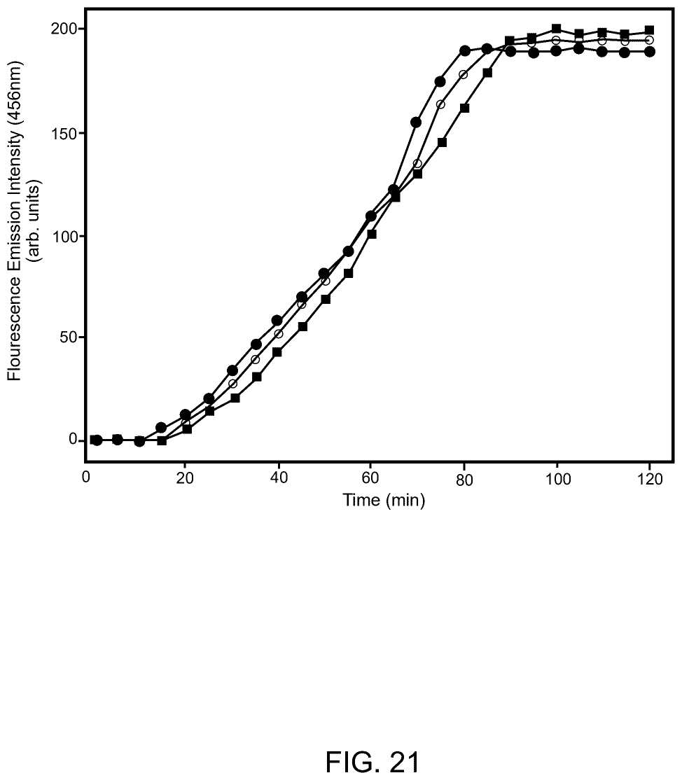

The enzymatic assay for fatty acids was used to measure the activity of the long-chain fatty acid CoA ligase reaction for the three different fatty acids (the reaction shown in FIG. 2). The time course of these reactions is shown in FIG. 20. Shown in FIG. 20 is a graph of the conversion for octanoic acid (C6), myristic acid (C12), and behenic acid (C20). Although the kinetics of the reaction were slightly different over the course of the assay, all reactions were complete within two hours under the conditions tested. Coincident with the loss of measurable fatty acid in the reaction is the formation of the pyrophosphate that is liberated in the formation of the long-chain fatty acid CoA molecules. Shown in FIG. 21 is a graph of the formation of pyrophosphate in the initial reaction. As shown in FIGS. 20 and 21, the time course of the pyrophosphate formation reaction completely mirrored the loss of fatty acid. Hence the key reaction in this disclosure has been clearly demonstrated for three fatty acids that span the available chain lengths.

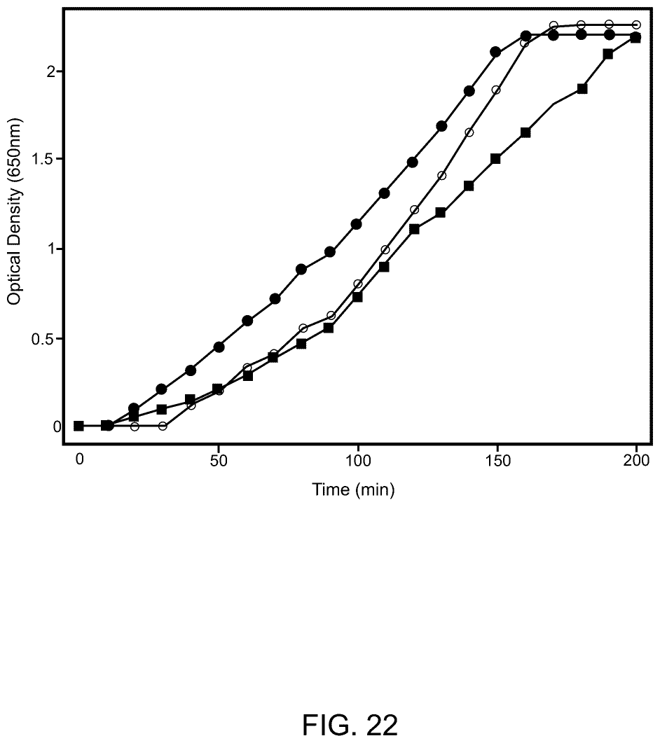

Second, once the long-chain fatty acid CoA moieties were produced, they were converted to PHAs through the five-step reaction shown in FIG. 3. The overall five-step reaction was easily monitored by the formation of PHA that precipitated from the solution, which is graphically shown in FIG. 22. FIG. 22 shows that there was a measurable increase in optical density due to light scattering (650 nm) after a lag of approximately 10 minutes, depending on which chain-length fatty acid was utilized as the initial substrate. The lag corresponds to the time it took for the first four enzymatic steps to be accomplished and for sufficient substrate to accumulate for the final reaction to take place. It is of note that the lag phase in this example was shorter than for similar reactions conducted under thermophilic reactions. Shorter lag phases are a hallmark of halophilic reactions, where enzymes generally have higher k_{cal}/Kₘ values.

The formation of precipitating PHA was then linear for the next 100 minutes, followed by the end of the reaction when octanoic acid and behenic acid were used as starting material or followed by a continued linear increase in PHA formation when myristic acid was used as the beginning substrate. Although the slopes of the linear portion of the reaction were nearly identical, it is not clear what was driving the behavior at the end of the assays, especially because there was no correlation between the overall kinetics of the reaction and the fatty acid chain-length. The main observation was that utilizing this scheme made it possible to efficiently produce precipitating material.

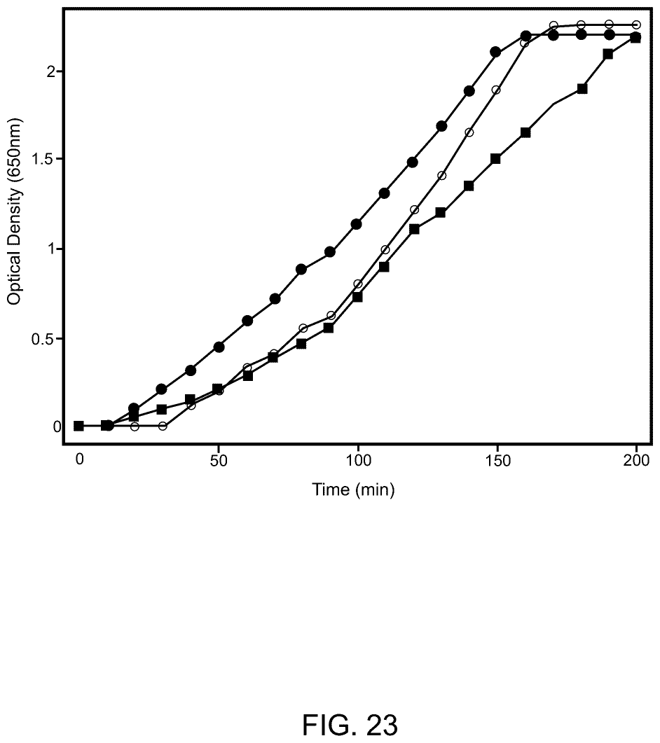

The PHA material formed using octanoic acid, myristic acid, or behenic acid was then depolymerized. FIG. 23 graphically shows the depolymerization of the formed PHA as measured by the decrease in optical density (due to scattering) at 650 nm. The myristic acid-based reaction was driven to near completion, as evidenced by near zero optical density at 60 minutes; however, the arachidic acid-based reaction ended with some undegraded material remaining. A possible explanation for the difference between these reactions could be kinetic and substrate preference differences by the enzyme for the three starting materials.

Sequences of the various enzymes used in the examples are provided in FIGS. 5A through 5D.

This exemplifies a multistep enzymatic process to create a variety of PHA bioplastic polymers from long-chain fatty acids or precursors thereof. Thus, the example illustrates the enzymatic conversion of long-chain fatty acids or precursors thereof to PHA monomers suitable for the production of bioplastic polymers.

These and other modifications and variations to the present invention may be practiced by those of ordinary skill in the art, without departing from the spirit and scope of the present invention, which is more particularly set forth in the appended claims. In addition, it should be understood that facets of the various aspects may be interchanged both in whole or in part. Furthermore, those of ordinary skill in the art will appreciate that the foregoing description is by way of example only, and is not intended to limit the invention so further described in such appended claims.

1. An enzymatic process for producing homogeneous chain length polyhydroxyalkanoate monomers and/or polymers having a chain length of at least eight carbons comprising:
contacting a long-chain fatty acid or precursor thereof, in vitro with an enzyme or a mixture of enzymes; and
producing homogeneous chain length polyhydroxyalkanoate monomers and/or polymers having a chain length of at least eight carbons.

2. The process of claim 1, wherein the homogeneous chain length polyhydroxyalkanoate monomers and/or polymers have a carbon chain length that is equal to a central carbon chain length of the long-chain fatty acid or precursor thereof.

3. The process of claim 1, wherein the central carbon chain length of the long chain fatty acid is any one of the following carbon chain lengths: C6, C7, C8, C9, C10, C11, C12, C13, C14, C15, C17, C18, C19, C20, C21, C22, C23, C24, C25, C26, C27, C28, C29, and C30, and/or wherein the central carbon chain length of the long-chain fatty acid precursor is configured to produce a long chain fatty acid having any one of the following central carbon chain lengths: C6, C7, C8, C9, C10, C11, C12, C13, C14, C15, C17, C18, C19, C20, C21, C22, C23, C24, C25, C26, C27, C28, C29, and C30.

4. The process of claim 1, wherein the process is carried out in one vessel, or wherein the process is carried out in more than one vessel.

5. The process of claim 1, wherein the long-chain fatty acid precursor is a long-chain alkane, a long-chain alcohol, or a long-chain aldehyde.

6. The process of claim 1, wherein the enzyme or mixture of enzymes contains at least one enzyme selected based upon the central chain length of the long-chain fatty acid.

7. The process of claim 1, further comprising a second long-chain fatty acid or precursor thereof having a different chain length from the long-chain fatty acid or precursor thereof, wherein the homogenous chain length polyhydroxyalkanoate comprises a copolymer having segments of the long-chain fatty acid or precursor thereof and second long-chain fatty acid or precursor thereof.

8. The process of claim 1, wherein the process further comprises one or more further steps of contacting the long chain fatty acid or precursor thereof in vitro with the enzyme or the mixture of enzymes.

9. The process of claim 1, wherein the process includes contacting the long chain fatty acid or precursor thereof in vitro with a first enzyme followed by one or more subsequent enzymes in a stepwise manner.

10. The process of claim 1, wherein the process includes contacting the long chain fatty acid or precursor thereof in vitro with two or more enzymes simultaneously.

11. The process of claim 1, wherein the homogeneous chain length polyhydroxyalkanoate monomers and/or polymers comprises any one or more of the following carbon chain lengths: C8, C9, C10, C11, C12, C13, C14, C15, C17, C18, C19, C20, C21, C22, C23, C24, C25, C26, C27, C28, C29, and C30.

12. The process of claim 1, wherein the homogeneous chain length polyhydroxyalkanoate monomers and/or polymers have a linear carbon chain.

13. The process of claim 1, wherein the homogeneous chain length polyhydroxy alkanoate monomers and/or polymers is at least about 90% homogeneous.

14. The process of claim 1, wherein the enzyme or the mixtures of enzymes is purified from an extremophilic microorganism, preferably wherein the microorganism is a bacteria of genera: Halomonas, Lihuaxuella, Lysobacter, Alteromonas, Arthrobacter, Azospirillum, Empedobacter, Desulfovibrio, Halobacillus, Halobacteriovorax, Haloechinothrix, Halomarina, Halorussus, Haloterrigena, Isoptericola, Marinobacter, Methyloligella, Micromonospora, Natronococcus, Nocardiopsis, Paracoccus, Roseivivax, Saccharomonospora, Shewanella, Alicyclobacillus, Natranaerobius, Halobacteriaceae, Hyphomonas, Amycolatopsis, Georgenia, Acidothermus, Thermobifida, or a combination thereof.

15. The process of claim 14, wherein the microorganism is an engineered microorganism that has been genetically modified to secrete a specific enzyme.

16. The process of claim 14, wherein the microorganism is at least one type of a naturally occurring microorganism that naturally encodes a specific enzyme.

17. The process of claim 1, wherein the enzyme or the mixtures of enzymes is a thermophilic enzyme.

18. The process of claim 17, wherein the thermophilic enzyme is temperature tolerant from about 40° C. to about 120° C.

19. The process of claim 1, wherein the process is performed at a temperature range from about 40° C. to about 80° C.

20. The process of claim 1, wherein the process comprises enzymes that co-function effectively in the same environment characterized by the same or similar pH and temperature.

21. The process of claim 1, where the process is an organism-free process, wherein the long-chain fatty alcohol or precursor thereof is contacted with the enzyme or mixture of enzymes in an environment substantially absent any bacteria that secrete the same enzyme or the mixture of the same enzymes.

22. An uncharacterized polyhydroxyalkanoate comprising:
a polyhydroxyalkanoate having a carbon chain length greater than C8, wherein the polyhydroxyalkanoate is a linear chain polymer substantially absent any side chain pendant polymers.

23. A process of producing homogeneous chain length polyhydroxyalkanoate monomers and/or polymers having a chain length of at least eight carbons comprising:
contacting a long-chain fatty acid with a long chain fatty acid CoA ligase/synthetase to obtain a long-chain fatty acid acyl-CoA;
contacting the long-chain fatty acid acyl-CoA with a long chain acyl-CoA dehydrogenase, followed by a long-chain-enoyl-CoA hydratase, followed by a hydroxyacyl-CoA dehydrogenase or a long-chain-3-hydroxyacyl-CoA dehydrogenase to obtain a long-chain acetoacetyl-CoA; and
contacting a long-chain acetoacetyl-CoA with an acetoacetyl-CoA reductase to obtain a hydroxyacyl-CoA for polymerization into a polyhydroxyalkanoate.

24. The process of claim 23, wherein the long chain fatty acid CoA ligase/synthetase is purified from Thermobifida halotolerans.

25. The process of claim 23, wherein the alcohol dehydrogenase is a fungal long-chain alcohol dehydrogenase purified from the group of fungal genera comprising: Aureobasidium, Macroventuria, Lophium, Tothia, Trichodelitschia, Westerdykella, Didymosphaeria, Viridothelium, Delitschia, Zopfia, Myriangium, Rhizodiscina, Saccharata, Aaosphaeria, Amniculicola, Byssothecium, Aspergillus, Meira, Dissoconium, Lizonia, Aureobasidium, Morchella, Sodiomyces, Tilletiopsis, Jaminaea, Ceraceosorus, Testicularia, Tilletiopsis, Violaceomyces, Rhizopus, Alternaria, Hesseltinella, Neurospora, Ramularia, and Rhynchosporium.
